(19) United States
(12) Patent Application Publication
(10) Pub. No.: US 2019/0185508 A1
(43) Pub. Date: Jun. 20, 2019

(54) DEUTERATED NUCLEOSIDE REVERSE TRANSCRIPTASE INHIBITORS

(71) Applicants: Merck Sharp & Dohme Corp., Rahway, NJ (US); Idenix Pharmaceuticals LLC, Cambridge, MA (US)

(72) Inventors: Francois-Rene Alexandre, Montpellier (FR); Izzat Raheem, Doylestown, PA (US)

(73) Assignees: Merck Sharp & Dohme Corp., Rahway, NJ (US); Idenix Pharmaceuticals LLC, Cambridge, MA (US)

(21) Appl. No.: 16/212,163

(22) Filed: Dec. 6, 2018

Related U.S. Application Data
(60) Provisional application No. 62/599,087, filed on Dec. 15, 2017.

(51) Int. Cl. C07H 19/23; A61K 31/7072; A61K 31/7076; A61K 31/7064; C07H 19/16; A61P 31/18

(52) U.S. Cl. CPC C07H 19/23; A61K 31/7072; A61P 31/18; A61K 31/7064; C07H 19/16; A61K 31/7076

(57) ABSTRACT

The present invention is directed to deuterated 4′-substituted nucleoside derivatives of Formula I

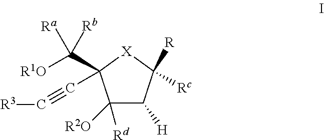

and their use in the inhibition of HIV reverse transcriptase, the prophylaxis of infection by HIV, the treatment of infection by HIV, and the prophylaxis, treatment, and delay in the onset or progression of AIDS and/or ARC.

### BACKGROUND OF THE INVENTION

The retrovirus designated human immunodeficiency virus (HIV), particularly the strains known as HIV type-1 (HIV-1) and type-2 (HIV-2), have been etiologically linked to the immunosuppressive disease known as acquired immunodeficiency syndrome (AIDS). HIV seropositive individuals are initially asymptomatic but typically develop AIDS related complex (ARC) followed by AIDS. Affected individuals exhibit severe immunosuppression which makes them highly susceptible to debilitating and ultimately fatal opportunistic infections. Replication of HIV by a host cell requires integration of the viral genome into the host cell's DNA. Since HIV is a retrovirus, the HIV replication cycle requires transcription of the viral RNA genome into DNA via an enzyme known as reverse transcriptase (RT).

Reverse transcriptase has three known enzymatic functions: The enzyme acts as an RNA-dependent DNA polymerase, as a ribonuclease, and as a DNA-dependent DNA polymerase. In its role as an RNA-dependent DNA polymerase, RT transcribes a single-stranded DNA copy of the viral RNA. As a ribonuclease, RT destroys the original viral RNA and frees the DNA just produced from the original RNA. And as a DNA-dependent DNA polymerase, RT makes a second, complementary DNA strand using the first DNA strand as a template. The two strands form double-stranded DNA, which is integrated into the host cell's genome by the integrase enzyme.

It is known that compounds that inhibit enzymatic functions of HIV RT will inhibit HIV replication in infected cells. These compounds are useful in the prophylaxis or treatment of HIV infection in humans. Among the compounds approved for use in treating HIV infection and AIDS are nucleoside RT inhibitors (NRTI) such as 3′-azido-3′-deoxythymidine (AZT), 2′,3′-dideoxyinosine (ddI), 2′,3′-dideoxycytidine (ddC), d4T, 3TC, abacavir, emtricitabine, and tenofovir disoproxil fumarate, as well as non-nucleoside RT inhibitors (nNRTI) such as nevirapine, delavirdine, and efavirenz.

While each of the foregoing drugs is effective in treating HIV infection and AIDS, there remains a need to develop additional HIV antiviral drugs including additional RT inhibitors.

A particular problem is the development of mutant HIV strains that are resistant to the known inhibitors. The use of anti-retrovirals to treat AIDS often leads to viruses that are less sensitive to the inhibitors. This resistance is typically the result of mutations that occur in the reverse transcriptase segment of the pol gene. The continued use of antiviral compounds to prevent HIV infection will inevitably result in the emergence of new resistant strains of HIV. Accordingly, there is a continuing need for new RT inhibitors that are effective against mutant HIV strains.

### SUMMARY OF THE INVENTION

The present invention is directed to deuterated 4′-substituted nucleoside derivatives and their use in the inhibition of HIV reverse transcriptase, the prophylaxis of infection by HIV, the treatment of infection by HIV, and the prophylaxis, treatment, and delay in the onset or progression of AIDS and/or ARC.

### DETAILED DESCRIPTION OF THE INVENTION

The present invention is directed to compounds having structural Formula I:

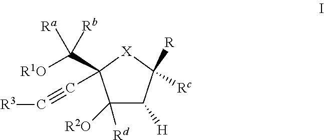

or a pharmaceutically acceptable salt thereof, wherein:
R is

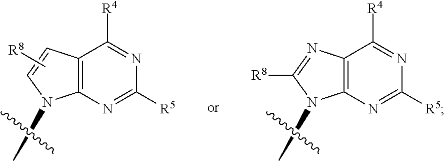

X is O, S, CH₂ or CF₂;
R¹ is —H, —C(O)R⁶, —C(O)OR⁶, —C(O)N(R⁶)₂;

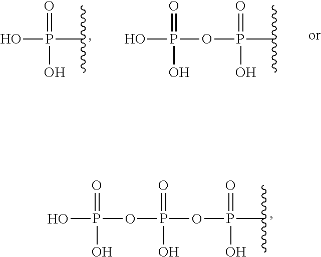 [SMILES: *P(=O)(O)O ; *P(=O)(O)OP(=O)(O)O ; *P(=O)(O)OP(=O)(O)OP(=O)(O)O]

or a pro-drug modification of the mono-, di- or triphosphate;
R² is —H, —C(O)R^{6a}, —C(O)OR⁶a or —C(O)N(R^{6a})₂;
R³, R^{a}, R^{b}, R^{c} and R^{d} are each independently —H or -D, provided that at least one of R³, R^{a}, R^{b}, R^{c} and R^{d} is -D;
D is deuterium (2H);
R⁴ is —H, —C₁-C₆ alkyl, —C₂-C₆ alkenyl, —C₂-C₆ alkynyl, —C₁-C₆ haloalkyl, —C₃-C₇ cycloalkyl, 5- or 6-membered monocyclic heteroaryl, a 9- or 10-membered bicyclic heteroaryl, halo, —CN, —NO₂, —N(R^{X})₂, —NH(C₁-C₆alkylene)-(5- or 6-membered monocyclic heteroaryl), —NH(C₁-C₆ alkylene)-(9- or 10-membered bicyclic heteroaryl), aryl, —NHC(O)OR^{6b}, —N(C(O)OR^{6b})₂, —NHC(O)N(R^{6b})₂, or —NHC(O)R^{6b}, wherein each of said —C₁-C₆ alkyl group, said —C₂-C₆ alkenyl group or said —C₂-C₆ alkynyl group can be optionally substituted with halo;
R⁵ is —H, —C₁-C₆ alkyl, —C₂-C₆ alkenyl, —C₂-C₆ alkynyl, —C₁-C₆ haloalkyl, —C₃-C₇ cycloalkyl, 5- or 6-membered monocyclic heteroaryl, a 9- or 10-membered bicyclic heteroaryl, halo, —OR^{X}, —CN, —NO₂, —N(R^{X})₂, —NH(C₁-C₆alkylene)-(5- or 6-membered monocyclic heteroaryl), —NH(C₁-C₆ alkylene)-(9- or 10-membered bicyclic heteroaryl), aryl, —NHC(O)OR^{6b}, —N(C(O)OR^{6b})₂, —NHC(O)N(R^{6b})₂, or —NHC(O)R^{6b}, wherein each of said —C₁-C₆ alkyl group, said —C₂-C₆ alkenyl group or said —C₂-C₆ alkynyl group can be optionally substituted with halo;
R⁶, R^{6a} and R^{6b} are each independently selected at each occurrence from —H, —C₁-C₆ alkyl, —C₁-C₆ haloalkyl, —(C₁-C₃ alkylene)ₘ-(C₃-C₇ cycloalkyl), —(C₁-C₃ alkylene)ₘ-(aryl), —(C₁-C₃ alkylene)ₘ-(4 to 7-membered heterocycloalkyl), —(C₁-C₃ alkylene)ₘ-(5- or 6-membered monocyclic heteroaryl) or —(C₁-C₃ alkylene)ₘ-(9- or 10-membered bicyclic heteroaryl), wherein each of said —C₁-C₆ alkyl, said C₃-C₇ cycloalkyl group, said aryl group, said 4 to 7-membered heterocycloalkyl group, said -(5- or 6-membered monocyclic heteroaryl group or said 9- or 10-membered bicyclic heteroaryl group can be optionally substituted with R⁷;
m is an integer selected from 0 (zero) or 1;
R⁷ represents from one to five substituent groups, each independently selected from —C₁-C₆alkyl, C₂-C₆ alkenyl, C₂-C₆ alkynyl, C₁-C₆ haloalkyl, aryl, or a 5-6-member heteroaryl;
R⁸ is —H, halo, —C₁-C₆ alkyl, —C₁-C₆ haloalkyl, —CN, —OR^{Y} or —N(R^{Y})₂;
R^{X} is independently selected at each occurrence from —H, —C₁-C₆ alkyl, —C₁-C₆ haloalkyl, aryl, or 5- or 6-membered monocyclic heteroaryl;
or when either or both of R⁴ or R⁵ is —N(R^{X})₂, each R^{X} may optionally be joined together with the nitrogen to which they are both attached to form a 5- or 6-membered monocyclic heteroaryl or 9- or 10-membered bicyclic heteroaryl; and
R^{Y} is —H, —C₁-C₆ alkyl or —C₁-C₆ haloalkyl.

In an embodiment of this invention referred to herein as Formula Ia are compounds of Formula I, or a pharmaceutically acceptable salt thereof, wherein R is

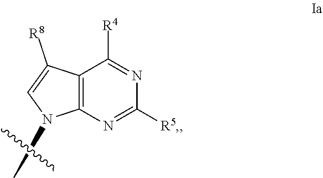

and all other variables therein (R¹, R², R³, etc.) are as defined in Formula I.

In another embodiment of this invention referred to herein as Formula Ib are compounds of Formula I, or a pharmaceutically acceptable salt thereof, wherein R is

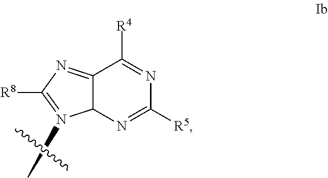

and all other variables therein (R¹, R², R³, etc.) are as defined in Formula I.

In another embodiment of this invention referred to herein as Embodiment A are compounds of Formula I, Ia or Ib, or a pharmaceutically acceptable salt thereof, wherein:

X is O, S or CH₂. In a class thereof, X is O.

In an embodiment of this invention referred to herein as Embodiment B are compounds of Formula I, Ia or Ib, Embodiment A or a class thereof, or a pharmaceutically acceptable salt thereof, wherein:

### (a) R¹ is —H; or

### (b) R¹ is

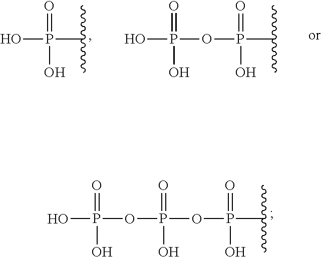 [SMILES: *P(=O)(O)O ; *P(=O)(O)OP(=O)(O)O ; *P(=O)(O)OP(=O)(O)OP(=O)(O)O]

or

### (c) R¹ is

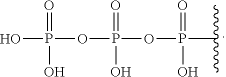 [SMILES: *P(=O)(O)OP(=O)(O)OP(=O)(O)O]

In an embodiment of this invention are compounds of Formula I, Ia or Ib, Embodiment A or B or a classes thereof, or a pharmaceutically acceptable salt thereof, wherein R² is —H.

In another embodiment of this invention are compounds of Formula I, Ia or Ib, or Embodiment A or B or classes thereof, or a pharmaceutically acceptable salt thereof, wherein R³ is -D.

In another embodiment of this invention are compounds of Formula I, Ia or Ib, or

Embodiment A or B or classes thereof, or a pharmaceutically acceptable salt thereof, wherein R^{a} is -D.

In another embodiment of this invention are compounds of Formula I, Ia or Ib, or Embodiment A or B or classes thereof, or a pharmaceutically acceptable salt thereof, wherein R^{b} is -D. In a class thereof, R^{a} and R^{b} are both -D.

In another embodiment of this invention are compounds of Formula I, Ia or Ib, or Embodiment A or B or classes thereof, or a pharmaceutically acceptable salt thereof, wherein R^{c} is -D.

In another embodiment of this invention are compounds of Formula I, Ia or Ib, or Embodiment A or B or classes thereof, or a pharmaceutically acceptable salt thereof, wherein R^{d} is -D.

In other embodiments of this invention are compounds of Formula I, Ia or Ib, or Embodiment A or B or classes thereof, or a pharmaceutically acceptable salt thereof, wherein:

R¹ is —H, —C(O)R⁶, —C(O)OR⁶, —C(O)N(R⁶)₂, or a pro-drug modification of

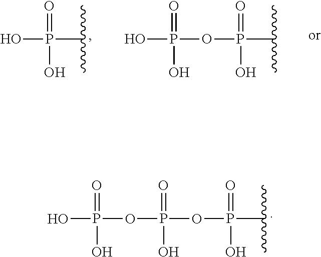 [SMILES: *P(=O)(O)O ; *P(=O)(O)OP(=O)(O)O ; *P(=O)(O)OP(=O)(O)OP(=O)(O)O]

In another embodiment of this invention are compounds of Formula I, Ia or Ib, or Embodiment A or B or classes thereof, or a pharmaceutically acceptable salt thereof, wherein R¹ is —H, —C(O)R⁶, —C(O)OR⁶, or —C(O)N(R⁶)₂; or a pro-drug modification of the mono-, di- or triphosphate. In a class thereof, R¹ is —C(O)R⁶, —C(O)OR⁶, or —C(O)N(R⁶)₂; or a pro-drug modification of the mono-, di- or triphosphate.

In other embodiments of this invention are compounds of Formula I, Ia or Ib, or Embodiment A or B or classes thereof, or a pharmaceutically acceptable salt thereof, wherein:

R² is —H, —C(O)R^{6a}, —C(O)OR⁶a or —C(O)N(R^{6a})₂;
or R² is —C(O)R^{6a}, —C(O)OR⁶a or —C(O)N(R^{6a})₂;

### or R² is —H.

In a further embodiment are compounds of Formula I, Ia or Ib, or Embodiment A or B or classes thereof, or a pharmaceutically acceptable salt thereof, wherein R³ is —H.

In another embodiment of this invention are compounds of Formula I, Ia or Ib, or Embodiment A or B, or a pharmaceutically acceptable salt thereof, wherein R⁴ is —N(R^{X})₂, —NHC(O)OR^{6b}, —N(C(O)OR^{6b})₂, —NHC(O)N(R^{6b})₂, or —NHC(O)R^{6b}; and particularly R⁴ is —N(R^{X})₂. More particularly R⁴ is —NH₂.

In other embodiments of this invention are compounds of Formula I, Ia or Ib, or Embodiment B, or a pharmaceutically acceptable salt thereof, wherein:

R⁵ is —H, halo, —C₁-C₆ alkyl, —OR^{X}, —N(R^{X})₂, —NHC(O)OR^{6b}, —N(C(O)OR^{6b})₂, —NHC(O)N(R^{6b})₂, or —NHC(O)R^{6b};
or R⁵ is —NHC(O)OR^{6b}, —N(C(O)OR^{6b})₂, —NHC(O)N(R^{6b})₂, or —NHC(O)R^{6b};
or R⁵ is —H, halo (preferably —F or —CO, —C₁-C₆ alkyl, —OR^{X} or —N(R^{X})₂;
or R⁵ is —H, —F or —NH₂.

In other embodiments of this invention are compounds of Embodiment A or B, or a pharmaceutically acceptable salt thereof, wherein:

R⁵ is —H, halo (preferably —F or —CO, —C₁-C₆ alkyl, —OR^{X} or —N(R^{X})₂;
or R⁵ is —H, —F or —NH₂.

In another embodiment of this invention are compounds of Formula I, Ia or Ib, or Embodiment B, or a pharmaceutically acceptable salt thereof, wherein R⁶, R^{6a} and R^{6b} are each independently selected at each occurrence from —H, —C₁-C₆ alkyl, —C₁-C₆ haloalkyl, aryl (preferably phenyl), or a 5- or 6-membered monocyclic heteroaryl. In another embodiment of this invention are compounds of Embodiment A, or a pharmaceutically acceptable salt thereof, wherein R^{6b} is selected at each occurrence from —H, —C₁-C₆ alkyl, —C₁-C₆ haloalkyl, aryl (preferably phenyl), or a 5- or 6-membered monocyclic heteroaryl.

In another embodiment of this invention are compounds of Formula I, Ia or Ib, or Embodiment A or B, or a pharmaceutically acceptable salt thereof, wherein R⁷ is —C₁-C₆ alkyl, aryl (preferably phenyl) or 5-6 member monocyclic heteroaryl.

In another embodiment of this invention are compounds of Formula I, Ia or Ib, or Embodiment A or B, or a pharmaceutically acceptable salt thereof, wherein R⁸ is —H, halo, —C₁-C₃ alkyl, —C₁-C₃ haloalkyl, —CN, —OR^{Y}, or —N(R^{Y})₂; and particularly it is —H, —F, —Cl, or —CH₃. In a class thereof, R⁸ is —H, halo or —CH₃.

In another embodiment, of this invention are compounds of Formula I or a pharmaceutically acceptable salt thereof, wherein;

### X is O;

### R¹ is —H;

### R² is —H;

R³, R^{a}, R^{b}, R^{c} and R^{d} are each independently —H or -D, provided that at least one of R³, R^{a}, R^{b}, R^{c} and R^{d} is -D;
D is deuterium;
R⁴ is —NH₂;
R⁵ is —H or halo; and
R⁸ is —H or halo.

All structural Formulas, embodiments and classes thereof described herein include the pharmaceutically acceptable salts of the compounds defined therein. Reference to the compounds of Formula I herein encompasses the compounds of each of Formulas I, Ia or Ib, and all embodiments and classes thereof. Reference to the compounds of this invention as those of a specific formula or embodiment, e.g., Formula I, Ia or Ib, or embodiments thereof, or any other generic structural formula or specific compound described or claimed herein, is intended to encompass the specific compound or compounds falling within the scope of the Formula or embodiment, including salts thereof, particularly pharmaceutically acceptable salts, solvates (including hydrates) of such compounds and solvated salt forms thereof, where such forms are possible, unless specified otherwise.

The present invention includes each of the Examples described herein, and pharmaceutically acceptable salts thereof. The invention also encompasses pharmaceutical compositions comprising an effective amount of a compound of the invention or a pharmaceutically acceptable salt thereof, and a pharmaceutically acceptable carrier.

As used herein, the term “alkyl” refers to a monovalent straight or branched chain, saturated aliphatic hydrocarbon radical having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and t-butyl, n- and iso-propyl, ethyl and methyl; “C₁₋₄ alkyl” (or “C₁-C₄ alkyl”) refers to n-, iso-, sec- and t-butyl, n- and iso-propyl, ethyl and methyl; and “C₁₋₃ alkyl” (or “C₁-C₃ alkyl”) refers to n- and iso-propyl, ethyl and methyl.

The term “alkenyl” refers to a monovalent straight or branched chain aliphatic hydrocarbon radical containing one carbon-carbon double bond and having a number of carbon atoms in the specified range. Thus, for example, “C₂₋₆ alkenyl” (or “C₂₋C₆ alkenyl”) refers to all of the hexenyl and pentenyl isomers as well as 1-butenyl, 2-butenyl, 3-butenyl, isobutenyl, 1-propenyl, 2-propenyl, and ethenyl (or vinyl).

The term “alkynyl” refers to a monovalent straight or branched chain aliphatic hydrocarbon radical containing one carbon-carbon triple bond and having a number of carbon atoms in the specified range. Thus, for example, “C₂₋₆ alkynyl” (or “C₂-C₆ alkynyl”) refers to all of the hexynyl and pentynyl isomers as well as 1-butynyl, 2-butynyl, 3-butynyl, 1-propynyl, 2-propynyl, and ethynyl.

The term “alkylene” refers to any divalent linear or branched chain aliphatic hydrocarbon radical having a number of carbon atoms in the specified range. Thus, for example, “—C₁₋₆ alkylene-” refers to any of the C₁ to C₆ linear or branched alkylenes, and “—C₁₋₃ alkylene-” refers to any of the C₁ to C₃ linear or branched alkylenes. For example, alkylene includes, but is not limited to, —(CH₂)₁₋₃—, —(CH₂)₂₋₃—, —(CH₂)₁₋₂—, —CH₂—, —CH(CH₃)—, and —C(CH₃)₂—.

The term “cycloalkyl” refers to any monocyclic ring of an alkane having a number of carbon atoms in the specified range. Thus, for example, “C₃₋₇ cycloalkyl” (or “C₃-C₇ cycloalkyl”) refers to cyclopropyl, cyclobutyl, cyclopentyl, cyclohexyl, and cycloheptyl. A particular class of interest for compounds of Formula I and embodiments thereof is C₃₋₆ cycloalkyl. The term “heterocycloalkyl” refers to a C₃₋₇ cycloalkyl ring wherein 1 or 2 of the carbons in the ring are replaced by N, O or S, independently selected at each point of replacement.

The term “halogen” (or “halo”) refers to fluorine, chlorine, bromine and iodine (alternatively referred to as fluoro, chloro, bromo, and iodo). A particular class of interest for compounds of Formula I and embodiments thereof is each of fluoro or chloro.

The term “haloalkyl” refers to an alkyl group as defined above in which one or more of the hydrogen atoms have been replaced with halo (i.e., —F, —Cl, —Br and/or —I). Thus, for example, “C₁₋₆ haloalkyl” (or “C₁-C₆ haloalkyl”) refers to a C₁ to C₆ linear or branched alkyl group as defined above with one or more halo substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.). A fluoroalkyl of particular interest is CF₃.

The term “C(O)” refers to carbonyl. The terms “S(O)₂” and “SO₂” each refer to sulfonyl. The term “S(O)” refers to sulfinyl.

The term “aryl” (or “C₆-C₁₀ aryl”) refers to (i) phenyl, or (ii) 9- or 10-membered bicyclic, fused carbocylic ring systems in which at least one ring is aromatic. Suitable aryls include, for example, phenyl, naphthyl, tetrahydronaphthyl (tetralinyl), or indenyl. In a particular class of compounds of Formula I and embodiments thereof, aryl is phenyl or naphthyl, and more particularly aryl is phenyl.

The term “heteroaryl” refers to (i) a 5- or 6-membered heteroaromatic ring containing from 1 to 4 heteroatoms independently selected from N, O and S, wherein each N is optionally in the form of an oxide to the extent chemically possible, (ii) a 9- or 10-membered bicyclic fused ring system, wherein the fused ring system contains from 1 to 6 heteroatoms independently selected from N, O and S, wherein each ring in the fused ring system contains zero, one, or more than one heteroatom, and at least one ring is aromatic, and each N is optionally in the form of an oxide to the extent chemically possible, and each S in a ring which is not aromatic is optionally S(O) or S(O)₂. Suitable 5- and 6-membered heteroaromatic rings include, for example, pyridyl, pyrrolyl, pyrazinyl, pyrimidinyl, pyridazinyl, triazinyl, thienyl, furanyl, imidazolyl, pyrazolyl, triazolyl triazolyl (i.e., 1,2,3-triazolyl or 1,2,4-triazolyl), tetrazolyl, oxazolyl, isooxazolyl, oxadiazolyl (i.e., the 1,2,3-, 1,2,4-, 1,2,5-(furazanyl), or 1,3,4-isomer), oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzopiperidinyl, benzisoxazolyl, benzoxazolyl, chromenyl, quinolinyl, isoquinolinyl, cinnolinyl, quinazolinyl, tetrahydroquinolinyl, tetrahydroisoquinolinyl, isoindolyl, benzodioxolyl (e.g., benzo-1,3-dioxolyl:

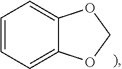

benzopiperidinyl, benzisoxazolyl, benzoxazolyl, chromanyl, isochromanyl, benzothienyl, benzofuranyl, imidazo[1,2-a]pyridinyl, benzotriazolyl, dihydroindolyl, dihydroisoindolyl, indazolyl, indolinyl, isoindolinyl, quinoxalinyl, quinazolinyl, 2,3-dihydrobenzofuranyl, and 2,3-dihydrobenzo-1,4-dioxinyl

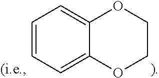

It is understood that the specific rings and ring systems suitable for use in the present invention are not limited to those listed in the preceding paragraphs. These rings and ring systems are merely representative. Unless expressly stated to the contrary in a particular context, any of the various cyclic rings and ring systems described herein may be attached to the rest of the compound at any ring atom (i.e., any carbon atom or any heteroatom) provided that the attachment is chemically allowed and a stable compound results.

Unless expressly depicted or described otherwise, a variable depicted in a structural formula with a “floating” bond attached to a ring, such as R⁸ in Formula I, is permitted to be a substituent on any available carbon or nitrogen atom in the ring to which the variable is attached.

When a moiety is noted as being “optionally substituted” in Formula I or any embodiment thereof, it means that Formula I or the embodiment thereof encompasses both compounds that are substituted with the noted substituent (or substituents) on the moiety and compounds that do not contain the noted substituent (or substituents) on the moiety (i.e., wherein the moiety is unsubstituted). As one example, when R⁴ is a —C₁-C₆ alkyl group that can be optionally substituted with halo, then R⁴ can be —C₁-C₆ alkyl or —C₁-C₆ haloalkyl.

When any variable (e.g., R⁷, R^{X}, R^{Y}) occurs more than one time in any constituent or in Formula I or in any other formula depicting and describing compounds of the present invention, its definition on each occurrence is independent of its definition at every other occurrence. Also, combinations of substituents and/or variables are permissible only if such combinations result in stable compounds. Unless expressly stated to the contrary, substitution by a named substituent is permitted on any atom in a ring (e.g., cycloalkyl, aryl, or heteroaryl) provided such ring substitution is chemically allowed and results in a stable compound.

As would be recognized by one of ordinary skill in the art, certain of the compounds of the present invention can exist as tautomers. All tautomeric forms of these compounds, whether isolated individually or in mixtures, are within the scope of the present invention. For example, in instances where an —OH substituent is permitted on a heteroaromatic ring and keto-enol tautomerism is possible, it is understood that the substituent might in fact be present, in whole or in part, in the oxo (═O) form.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject). The compounds of the present invention are limited to stable compounds embraced by Formula I and its embodiments.

The compounds of Formula I may have one or more chiral (asymmetric) centers. The present invention encompasses all stereoisomeric forms of the compounds of Formula I. Centers of asymmetry that are present in the compounds of Formula I can all independently of one another have (R) or (5) configuration. When bonds to a chiral center are depicted as straight lines in the structural Formulas of the invention, or when a compound name is recited without an (R) or (5) chiral designation for a chiral center, it is understood that both the (R) and (5) configurations of each such chiral cener, and hence each enantiomer or diastereomer and mixtures thereof, are embraced within the Formula or by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis. The present invention includes all such isomers, as well as salts, solvates (which includes hydrates) and solvated salts of such racemates, enantiomers, diastereomers and tautomers and mixtures thereof.

The compounds can be administered in the form of pharmaceutically acceptable salts. The term “pharmaceutically acceptable salt” refers to a salt which is not biologically or otherwise undesirable (e.g., is neither toxic nor otherwise deleterious to the recipient thereof). When the compounds of Formula I contain one or more acidic or basic groups the invention also includes the corresponding pharmaceutically acceptable salts. Thus, the compounds of Formula I which contain acidic groups (e.g., —COOH or a phenolic group) can be used according to the invention as, for example but not limited to, alkali metal salts, alkaline earth metal salts or as ammonium salts. Examples of such salts include but are not limited to sodium salts, potassium salts, calcium salts, magnesium salts or salts with ammonia or organic amines such as, for example, ethylamine, ethanolamine, triethanolamine or amino acids. Compounds of Formula I which contain one or more basic groups, i.e. groups which can be protonated, can be used according to the invention in the form of their acid addition salts with inorganic or organic acids as, for example but not limited to, salts with hydrogen chloride, hydrogen bromide, phosphoric acid, sulfuric acid, nitric acid, benzenesulfonic acid, methanesulfonic acid, p-toluenesulfonic acid, naphthalenedisulfonic acids, oxalic acid, acetic acid, trifluoroacetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, etc. If the compounds of Formula I simultaneously contain acidic and basic groups in the molecule the invention also includes, in addition to the salt forms mentioned, inner salts or betaines (zwitterions). Salts can be obtained from the compounds of Formula I by customary methods which are known to the person skilled in the art, for example by combination with an organic or inorganic acid or base in a solvent or dispersant, or by anion exchange or cation exchange from other salts. The present invention also includes all salts of the compounds of Formula I which, owing to low physiological compatibility, are not directly suitable for use in pharmaceuticals but which can be used, for example, as intermediates for chemical reactions or for the preparation of pharmaceutically acceptable salts.

Another embodiment of the present invention is a compound of Formula I wherein the compound or its salt is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt. %) of a product containing a compound of Formula I or its salt (e.g., the product isolated from a reaction mixture affording the compound or salt) consists of the compound or salt. The level of purity of the compounds and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest purity level governs. A compound or salt of 100% purity is one which is free of detectable impurities as determined by a standard method of analysis. With respect to a compound of the invention which has one or more asymmetric centers and can occur as mixtures of stereoisomers, a substantially pure compound can be either a substantially pure mixture of the stereoisomers or a substantially pure individual diastereomer or enantiomer.

Furthermore, compounds of the present invention may exist in amorphous form and/or one or more crystalline forms, and as such all amorphous and crystalline forms and mixtures thereof of the compounds of Formula I are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms.

The compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof, pro-drug forms thereof which include any combination of stereoisomer, tautomer, solvate, hydrate, salt and/or physical forms of said pro-drugs, where such forms are possible unless specified otherwise.

Prodrug modification of the compounds of the invention are contemplated herein. Prodrugs of compounds of Formula I, or pharmaceutically acceptable salts thereof, can exhibit enhanced solubility, absorption, and/or lipophilicity compared to the compounds per se, thereby resulting in increased bioavailability and efficacy. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The in vivo transformation of a prodrug may occur by various mechanisms, e.g., an enzyme-catalyzed chemical reaction, a metabolic chemical reaction, and/or a spontaneous chemical reaction (e.g., solvolysis), such as, for example, through hydrolysis in blood. This invention encompasses any prodrugs which convert, due to intracellular/in vivo conversion, to a 4′-substituted nucleoside derivative of a compound of Formula I which is an inhibitor of HIV reverse transcriptase. For example, the term “prodrug” as it relates to the compounds of Formula I, includes a compound (e.g., a drug precursor), which may be in the form of a pharmaceutically acceptable salt, that is transformed intracellularly/in vivo to provide a compound of Formula I wherein R¹ is

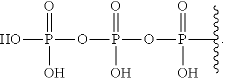 [SMILES: *P(=O)(O)OP(=O)(O)OP(=O)(O)O]

It is understood that a compound of Formula I or a salt thereof, wherein, for example, R¹ is (1) —H, (2) a pro-drug modification of the tri-phosphate; or

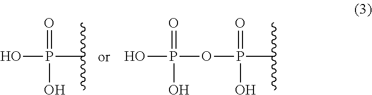 [SMILES: *P(=O)(O)O ; *P(=O)(O)OP(=O)(O)O]

or a prodrug modification of the mono- or di-phosphate;

may be converted intracellularly/in vivo by one or more mechanisms (e.g., enzyme-catalyzed chemical reactions) to the corresponding 5′ triphosphate

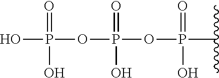 [SMILES: *P(=O)(O)OP(=O)(O)OP(=O)(O)O]

(i.e., wherein R¹ is —P(O)(OH)—O—P(O)(OH)—O—P(O)(OH)₂). While not wishing to be bound by any particular theory, the 5′triphosphate is generally understood to be responsible for inhibiting the HIV RT enzyme and for the resulting antiviral activity after administration of the compound of Formula I to a subject.

The term “pro-drug modification of the mono-, di- or triphosphate” as used herein includes, but is not limited to, 5′-alcohol-derived prodrugs such as —P(O)(—O—C₁-C₆alkyl)₂; —P(O)(—NH-(α-aminoacyl group))(—O-aryl), known as “McGuigan” type prodrugs, for example but not limited to, a compound of Formula I wherein R¹ is

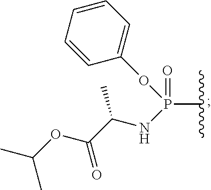 [SMILES: *P(=O)(N[C@@H](C)C(=O)OC(C)C)Oc1ccccc1]

—P(O)(—O—(C₁-C₆ alkylene)-S-acyl)(—NH-arylalkyl); S-acyl-2-thioethyl (SATE) prodrugs; a cyclic phosphate ester that forms a bridge between two hydroxyl groups, such as:

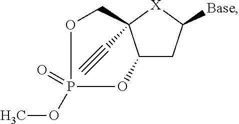

wherein the cyclic phosphate ester forms a bridge between the 3′-OH group and 5′-OH groups; and those described in U.S. Pat. No. 7,879,815; International Publication Nos. WO2005/003047, WO2008/082602, WO2010/0081628, WO2010/075517 and WO2010/075549; Mehellou, Chem. Med. Chem., 5:1841-1842 (2005); Bobeck et al., Antiviral Therapy 15:935-950 (2010); Furman et al., Future Medicinal Chemistry, 1:1429-1452 (2009); and Erion, Microsomes and Drug Oxidations, Proceedings of the International Symposium, 17th, Saratoga Springs, N.Y., United States, Jul. 6-10, 2008, 7-12 (2008).

Prodrug modification of the compounds of Formula I at positions other than the 5′-alcohol-derived prodrugs are also contemplated herein. When the compound contains, for example, a hydroxy group, the prodrug can be a derivative of the hydroxy group such as an ester (—OC(O)R), a carbonate ester (—OC(O)OR), a phosphate ester (—O—P(═O)(OH)₂), an ether (—OR), or a mono-phosphate prodrug such as a phosphoramidate (can be converted in vivo to the corresponding nucleoside monophosphate). Additionally, if a compound of Formula I contains an alcohol functional group, a prodrug can be formed by the replacement of one or more of the hydrogen atoms of the alcohol groups with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, or glycosyl (the radical resulting from the removal of a hydroxyl group of the hemiacetal form of a carbohydrate).

If the compound of Formula I incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N— or di-N,N(C₁-C₆)alkylaminoalkyl; —C(Y⁴)Y⁵ wherein Y⁴ is H or methyl and Y⁵ is mono-N— or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aryl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Other examples include the following: when the compound of Formula I contains a carboxylic acid group, the prodrug can be an ester or an amide, and when the compound of Formula I contains a primary amino group or another suitable nitrogen that can be derivatized, the prodrug can be an amide, carbamate, urea, imine, or a Mannich base. Conventional procedures for the selection and preparation of suitable prodrug derivatives are described, for example, in Design of Prodrugs, edited by H. Bundgaard, Elsevier, 1985; J. J. Hale et al., J. Med. Chem. 2000, vol. 43, pp. 1234-1241; C. S. Larsen and J. Ostergaard, “Design and application of prodrugs” in: Textbook of Drug Design and Discovery, 3^{rd} edition, edited by C. S. Larsen, 2002, pp. 410-458; and Beaumont et al., Current Drug Metabolism 2003, vol. 4, pp. 461-458; the disclosures of each of which are incorporated herein by reference in their entireties.

The invention also encompasses methods for the treatment or prophylaxis of infection by HIV, for the inhibition of HIV reverse transcriptase, for the treatment, prophylaxis, or delay in the onset of AIDS in a subject in need thereof, which comprises administering to the subject an effective amount of a compound of the invention or a pharmaceutically acceptable salt thereof.

The invention also encompasses a compound of the invention, or a pharmaceutically acceptable salt thereof, for use in the preparation of a medicament for the treatment or prophylaxis of infection by HIV, for the inhibition of HIV reverse transcriptase, or for the treatment, prophylaxis, or delay in the onset of AIDS in a subject in need thereof.

The invention also encompasses a pharmaceutical composition comprising an effective amount of a compound of the invention, or a pharmaceutically acceptable salt thereof, and a pharmaceutically acceptable carrier and further comprising an effective amount of an additional anti-HIV agent selected from the group consisting of HIV antiviral agents, immunomodulators, and anti-infective agents. Within this embodiment, the anti-HIV agent is an antiviral selected from the group consisting of HIV protease inhibitors, HIV reverse transcriptase inhibitors, HIV integrase inhibitors, HIV fusion inhibitors, HIV entry inhibitors, and HIV maturation inhibitors.

Compounds of Formula Ia or Ib, each form a subset of the compounds included in Formula I. Any description above or which follows that refers to a compound of Formula I also applies to a compound of each of Formula Ia or Ib, and each embodiment thereof.

Other embodiments of the present invention include the following:

(a) A pharmaceutical composition comprising an effective amount of a compound of Formula I as defined above, or a prodrug or pharmaceutically acceptable salt thereof, and a pharmaceutically acceptable carrier.

(b) A pharmaceutical composition which comprises the product prepared by combining (e.g., mixing) an effective amount of a compound of Formula I as defined above, or a prodrug or pharmaceutically acceptable salt thereof, and a pharmaceutically acceptable carrier.

(c) The pharmaceutical composition of (a) or (b), further comprising an effective amount of one or more an anti-HIV agents selected from the group consisting of HIV antiviral agents, immunomodulators, and anti-infective agents.

(d) The pharmaceutical composition of (c), wherein the anti-HIV agent is selected from one or more of an antiviral selected from the group consisting of HIV protease inhibitors, nucleoside HIV reverse transcriptase inhibitors, non-nucleoside HIV reverse transcriptase inhibitors, HIV integrase inhibitors, HIV fusion inhibitors, HIV entry inhibitors and HIV maturation inhibitors.

(e) A combination which is (i) a compound of Formula I as defined above, or a prodrug or pharmaceutically acceptable salt thereof, and (ii) an anti-HIV agent selected from the group consisting of HIV antiviral agents, immunomodulators, and anti-infective agents; wherein the compound and the anti-HIV agent are each employed in an amount that renders the combination effective for inhibition of HIV reverse transcriptase, for treatment or prophylaxis of infection by HIV, or for treatment, prophylaxis of, or delay in the onset or progression of AIDS.

(f) The combination of (e), wherein the anti-HIV agent is an antiviral selected from the group consisting of HIV protease inhibitors, nucleoside HIV reverse transcriptase inhibitors, non-nucleoside HIV reverse transcriptase inhibitors, HIV integrase inhibitors, HIV fusion inhibitors, HIV entry inhibitors and HIV maturation inhibitors.

(g) A method for the inhibition of HIV reverse transcriptase in a subject in need thereof which comprises administering to the subject an effective amount of a compound of Formula I or a prodrug or pharmaceutically acceptable salt thereof.

(h) A method for the prophylaxis or treatment of infection by HIV (e.g., HIV-1) in a subject in need thereof which comprises administering to the subject an effective amount of a compound of Formula I or a prodrug or pharmaceutically acceptable salt thereof.

(i) The method of (h), wherein the compound of Formula I is administered in combination with an effective amount of at least one other HIV antiviral selected from the group consisting of HIV protease inhibitors, HIV integrase inhibitors, non-nucleoside HIV reverse transcriptase inhibitors, nucleoside HIV reverse transcriptase inhibitors, HIV fusion inhibitors, HIV entry inhibitors and HIV maturation inhibitors.

(j) A method for the prophylaxis, treatment or delay in the onset or progression of AIDS in a subject in need thereof which comprises administering to the subject an effective amount of a compound of Formula I or a prodrug or pharmaceutically acceptable salt thereof.

(k) The method of (j), wherein the compound is administered in combination with an effective amount of at least one other HIV antiviral selected from the group consisting of HIV protease inhibitors, HIV integrase inhibitors, non-nucleoside HIV reverse transcriptase inhibitors, nucleoside HIV reverse transcriptase inhibitors, HIV fusion inhibitors, HIV entry inhibitors and HIV maturation inhibitors.

(l) A method for the inhibition of HIV reverse transcriptase in a subject in need thereof which comprises administering to the subject the pharmaceutical composition of (a), (b), (c) or (d) or the combination of (e) or (f).

(m) A method for the prophylaxis or treatment of infection by HIV (e.g., HIV-1) in a subject in need thereof which comprises administering to the subject the pharmaceutical composition of (a), (b), (c) or (d) or the combination of (e) or (f).

(n) A method for the prophylaxis, treatment, or delay in the onset or progression of AIDS in a subject in need thereof which comprises administering to the subject the pharmaceutical composition of (a), (b), (c) or (d) or the combination of (e) or (f).

The present invention also includes a compound of Formula I or pharmaceutically acceptable salt thereof, (i) for use in, (ii) for use as a medicament for, or (iii) for use in the preparation of a medicament for: (a) therapy (e.g., of the human body), (b) medicine, (c) inhibition of HIV reverse transcriptase, (d) treatment or prophylaxis of infection by HIV, or (e) treatment, prophylaxis of, or delay in the onset or progression of AIDS. In these uses, the compounds of the present invention can optionally be employed in combination with one or more anti-HIV agents selected from HIV antiviral agents, anti-infective agents, and immunomodulators.

Additional embodiments of the invention include the pharmaceutical compositions, combinations and methods set forth in (a)-(n) above and the uses (i)(a)-(e) through (iii)(a)-(e) set forth in the preceding paragraph, wherein the compound of the present invention employed therein is a compound of one of the embodiments, aspects, classes, sub-classes, or features described above. In all of these embodiments etc., the compound may optionally be used in the form of a prodrug or pharmaceutically acceptable salt or pharmaceutically acceptable salt of a prodrug.

Additional embodiments of the present invention include each of the pharmaceutical compositions, combinations, methods and uses set forth in the preceding paragraphs, wherein the compound of the present invention or its salt employed therein is substantially pure. With respect to a pharmaceutical composition comprising a compound of Formula I or its prodrug or salt and a pharmaceutically acceptable carrier and optionally one or more excipients, it is understood that the term “substantially pure” is in reference to a compound of Formula I or its prodrug and/or salt per se.

Still additional embodiments of the present invention include the pharmaceutical compositions, combinations and methods set forth in (a)-(n) above and the uses (i)(a)-(e) through (iii)(a)-(e) set forth above, wherein the HIV of interest is HIV-1. Thus, for example, in the pharmaceutical composition (d), the compound of Formula I is employed in an amount effective against HIV-1 and the anti-HIV agent is an HIV-1 antiviral selected from the group consisting of HIV-1 protease inhibitors, HIV-1 reverse transcriptase inhibitors, HIV-1 integrase inhibitors, HIV-1 fusion inhibitors and HIV-1 entry inhibitors. The compounds of Formula I may also be useful agents against HIV-2.

The term “administration” and variants thereof (e.g., “administering” a compound) in reference to a compound of Formula I means providing the compound to the individual in need of treatment or prophylaxis and includes both self-administration and administration to the patient by another person. When a compound or a prodrug thereof is provided in combination with one or more other active agents (e.g., antiviral agents useful for treating or prophylaxis of HIV infection or AIDS), “administration” and its variants are each understood to include provision of the compound or prodrug and other agents at the same time or at different times. When the agents of a combination are administered at the same time, they can be administered together in a single composition or they can be administered separately.

As used herein, the term “composition” is intended to encompass a product comprising the specified ingredients, as well as any product which results from combining the specified ingredients. Ingredients suitable for inclusion in a pharmaceutical composition are pharmaceutically acceptable ingredients. By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means an amount sufficient to inhibit HIV reverse transcriptase, inhibit HIV replication, exert a prophylactic effect, and/or a exert a therapeutic effect after administration. One embodiment of “effective amount” is a “therapeutically effective amount” which is an amount of a compound that is effective for inhibiting HIV reverse transcriptase, inhibiting HIV replication (either of the foregoing which may also be referred to herein as an “inhibition effective amount”), treating HIV infection, treating AIDS, delaying the onset of AIDS, and/or slowing progression of AIDS in a patient. Another embodiment of “effective amount” is a “prophylactically effective amount” which is an amount of the compound that is effective for prophylaxis of HIV infection or prophylaxis of AIDS in a patient. It is understood that an effective amount can simultaneously be both a therapeutically effective amount, e.g., for treatment of HIV infection, and a prophylactically effective amount, e.g., for prevention or reduction of risk for developing AIDS. When the compound of Formula I is administered as a salt, reference to an amount of the compound is to the free form (i.e., the non-salt form) of the compound.

In the method of the present invention (i.e., inhibiting HIV reverse transcriptase, treating or prophylaxis of HIV infection, inhibiting HIV replication, treating or prophylaxis of AIDS, delaying the onset of AIDS, or delaying or slowing progression of AIDS), the compounds of this invention, optionally in the form of a salt, can be administered by any means that produces contact of the active agent with the agent's site of action. They can be administered by conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically acceptable carriers, adjuvants and vehicles, any of which administration methods can be provided as a single dose, once-daily, or less frequently such as once weekly or once monthly in, for example but not limited to, the dosage ranges and amounts described below. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18th edition, edited by A. R. Gennaro, Mack Publishing Co., 1990 and in Remington—The Science and Practice of Pharmacy, 22nd Edition, published by Pharmaceutical Press and Philadelphia College of Pharmacy at University of the Sciences, 2012, ISBN 978 0 85711-062-6 and prior editions.

Formulations of compounds described by Formula I that result in drug supersaturation and/or rapid dissolution may be utilized to facilitate oral drug absorption. Formulation approaches to cause drug supersaturation and/or rapid dissolution include, but are not limited to, nanoparticulate systems, amorphous systems, solid solutions, solid dispersions, and lipid systems. Such formulation approaches and techniques for preparing them are well known in the art. For example, solid dispersions can be prepared using excipients and processes as described in reviews (e.g., A. T. M. Serajuddin, J Pharm Sci, 88:10, pp. 1058-1066 (1999)). Nanoparticulate systems based on both attrition and direct synthesis have also been described in reviews such as Wu et al (F. Kesisoglou, S. Panmai, Y. Wu, Advanced Drug Delivery Reviews, 59:7 pp. 631-644 (2007)).

The compounds of Formula I, and pharmaceutically acceptable salts thereof, are HIV reverse transcriptase inhibitors. The compounds are useful for inhibiting HIV reverse transcriptase and for inhibiting HIV replication in vitro and in vivo. More particularly, the compounds of Formula I inhibit the polymerase function of HIV-1 reverse transcriptase. The testing of compounds of the Examples of the invention in the assay set forth in the RT Polymerase Assay below, illustrates the ability of compounds of the invention to inhibit the RNA-dependent DNA polymerase activity of HIV-1 reverse transcriptase. The compounds of Formula I may also be useful agents against HIV-2.

The compounds of Formula I can be administered in a dosage range of 0.001 to 1000 mg/kg of mammal (e.g., human) body weight per day, or at other time intervals as appropriate, in a single dose or in divided doses. One example of a dosage range is 0.01 to 500 mg/kg body weight per day, or at other time intervals as appropriate, administered orally or via other routes of administration in a single dose or in divided doses. Another example of a dosage range is 0.1 to 100 mg/kg body weight per day, or at other time intervals as appropriate, administered orally or via other routes of administration in single or divided doses. For oral (e.g., tablets or capsules) or other routes of administration, the compositions can be provided containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. In some cases, depending on the potency of the compound or the individual response, it may be necessary to deviate upwards or downwards from the given dose. Compounds of the invention can be administered as a single dose, once-daily, or less frequently such as once weekly or once monthly in, for example but not limited to, the dosage ranges and amounts noted above. Furthermore, the compound may be formulated for immediate or modified release such as extended or controlled release.

As noted above, the present invention is also directed to use of a compound of Formula I with one or more anti-HIV agents. An “anti-HIV agent” is any agent which is directly or indirectly effective in the inhibition of HIV, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, imunomodulators, antiinfectives, or vaccines useful for treating HIV infection or AIDS. Suitable HIV antivirals for use in combination with the compounds of the present invention include, for example, those listed in Table A as follows:

TABLE A
Antiviral Agents for Treating HIV infection or AIDS

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| capravirine | nnRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| dolutegravir, Tivicay ® | InI |
| doravirine, MK-1439 | nnRTI |
| efavirenz, EFV, Sustiva ®, Stocrin ® | nnRTI |
| efavirenz + emtricitabine + tenofovir DF, Atripla ® | nnRTI + nRTI |
| EFdA (4′-ethynyl-2-fluoro-2′-deoxyadenosine) | nRTI |
| Elvitegravir | InI |
| emtricitabine, FTC, Emtriva ® | nRTI |
| emtricitabine + tenofovir DF, Truvada ® | nRTI |
| emivirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| PPL-100 (also known as PL-462) (Ambrilia) | PI |
| raltegravir, MK-0518, Isentress ™ | InI |
| Rilpivirine | nnRTI |
| ritonavir, Norvir ® | PI |
| saquinavir, Invirase ®, Fortovase ® | PI |
| stavudine, d4T,didehydrodeoxythymidine, Zerit ® | nRTI |
| tenofovir DF (DF = disoproxil fumarate), TDF, Viread ® | nRTI |
| Tenofovir, hexadecyloxypropyl (CMX-157) | nRTI |
| Tenofovir alafenamide fumarate (GS-7340) | nRTI |
| tipranavir, Aptivus ® | PI |
| Vicriviroc | EI |

or a pharmaceutically acceptable salt thereof.
   EI=entry inhibitor; FI=fusion inhibitor; InI=integrase inhibitor; PI=protease inhibitor; nRTI=nucleoside reverse transcriptase inhibitor; nnRTI=non-nucleoside reverse transcriptase inhibitor. Some of the drugs listed in the table are used in a salt form; e.g., abacavir sulfate, delavirdine mesylate, indinavir sulfate, atazanavir sulfate, nelfinavir mesylate, saquinavir mesylate, vicriviroc maleate.

It is understood that the scope of combinations of the compounds of this invention with anti-HIV agents is not limited to the HIV antivirals listed in Table A, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PDR, Thomson PDR, 57th edition (2003), the 58th edition (2004), or the 59th edition (2005) and the current Physicians' Desk Reference (68th ed.). (2014), Montvale, N.J.: PDR Network. The dosage ranges for a compound of the invention in these combinations can be the same as those set forth above.

The compounds of this invention are also useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV reverse transcriptase, e.g., by competitive inhibition.

The following abbreviations and acronyms may be used herein:

| | |
|---|---|
| Ac | acetyl |
| Ac₂O | acetic anhydride |
| ACN | acetonitrile |
| AcOH or HOAc | acetic acid |
| APCI | atmospheric-pressure chemical ionization |
| aq | aqueous |
| Bn | benzyl |
| Boc or BOC | tert-butoxycarbonyl |
| Bz | benzoyl |
| Cbz | benzyloxycarbonyl |
| calc'd | calculated |
| Celite | diatomaceous earth |
| DBU | 1,8-diazabicyclo(5.4.0)undec-7-ene |
| DCM | dichloromethane |
| DIEA or DIPEA | N,N-diisopropylethylamine |
| DMAP | 4-dimethylaminopyridine |
| DMF | N,N-dimethylformamide |
| DMSO | dimethyl sulfoxide |
| dMTrCl | 4,4′-dimethoxytrityl chloride |
| EDCI | 1-ethyl-3-(3-dimethylaminopropyl)carbodiimide |
| EDTA | ethylenediamine tetraacetic acid |
| ESI | electrospray ionization |
| Et | ethyl |
| Et₂O | diethyl ether |
| EtOH | ethanol |
| EtOAc | ethyl acetate |
| Et₃N | triethylamine |
| h | hour |
| HPLC | high-performance liquid chromatography |
| IBX | 2-iodoxybenzoic acid |
| IPA | isopropanol |
| iPr | isopropyl |
| LC | liquid chromatography |
| LCMS | liquid chromatography mass spectrometry |
| Me | Methyl |
| MeCN | acetonitrile |
| MeOH | methanol |
| mg | milligrams |
| min | minutes |
| □L | microliters |
| mL | milliliters |
| mmol | millimoles |
| MS | mass spectrometry |
| MTBE | methyl tert-butyl ether |
| MTS | (3-(4,5-dimethylthiazol-2-yl)-5- |
| | (3-carboxymethoxyphenyl)- |
| | 2-(4-sulfophenyl)-2H-tetrazolium) |
| NMR | nuclear magnetic resonance spectroscopy |
| PDA | photodiode array |
| PE | petroleum ether |
| Ph | phenyl |
| Pr | Propyl |
| PS | Polystyrene |
| Rac | racemic mixture |
| RT or rt | room temperature |
| Rt | retention time |
| Sat | saturated |
| SFC | supercritical fluid chromatography |
| TBAF | tert-butyl ammonium fluoride |
| TBS or TBDMS | tert-butyldimethylsilyl |
| TBSCl | tert-butyldimethylsilyl chloride |
| t-Bu | tert-butyl |
| TEA | triethylamine |
| TBDPS | tert-butyldiphenylsilyl |
| TBDPSCl | tert-butyldiphenylsilyl chloride |
| TFA | trifluoroacetic acid |
| THF | tetrahydrofuran |
| TLC | thin layer chromatography |
| TMS | trimethylsilyl |
| Tris | tris(hydroxymethyl)aminomethane |
| UPLC | ultra-performance liquid chromatography |

The invention is illustrated by the following examples. For all of the examples, standard work-up and purification methods known to those skilled in the art can be utilized. Unless otherwise indicated, all temperatures are expressed in ° C. (degrees Centigrade). All reactions are conducted at room temperature unless otherwise noted. Synthetic methodologies illustrated herein are intended to exemplify the applicable chemistry through the use of specific examples and are not indicative of the scope of the disclosure.

### General Procedures

Reactions sensitive to moisture or air were performed under nitrogen or argon atmosphere using anhydrous solvents and reagents. The progress of reactions was determined using either analytical thin layer chromatography (TLC) usually performed with E. Merck pre-coated TLC plates, silica gel 60E-254, layer thickness 0.25 mm or liquid chromatography-mass spectrometry (LC-MS).

The analytical UPLC-MS system used consisted of a Waters SQD2 platform with electrospray ionization in positive and negative detection mode with an Acquity UPLC I-class solvent manager, column manager, sample manager and PDA detector. The column used for standard methods was a CORTECS UPLC C18 1.6 μm, 2.1×30 mm, and the column used for polars method was an ACQUITY UPLC HSST3 1.8 μm, 2.1×30 mm, the column temperature was 40° C., the flow rate was 0.7 mL/min, and injection volume was 1 μL. UV detection was in the range 210-400 nm. The mobile phase consisted of solvent A (water plus 0.05% formic acid) and solvent B (acetonitrile plus 0.05% formic acid) with different gradients for 4 different methods: 1/Starting with 99% solvent A for 0.2 minutes changing to 98% solvent B over 1 minutes, maintained for 0.4 minutes, then reverting to 99% solvent A over 0.1 min; 2/Starting with 99% solvent A for 0.5 minutes changing to 98% solvent B over 3.7 minutes, maintained for 0.4 minutes, then reverting to 99% solvent A over 0.1 min; 3/Starting with 100% solvent A for 0.4 minutes changing to 98% solvent B over 0.9 minutes, maintained for 0.3 minutes, then reverting to 100% solvent A over 0.1 min; 4/Starting with 100% solvent A for 0.8 minutes changing to 98% solvent B over 3.4 minutes, maintained for 0.4 minutes, then reverting to 100% solvent A over 0.1 minutes.

The analytical LC-MS system used consisted of a Agilent 6140 quadrupole LC/MS platform with electrospray ionization in positive and negative detection mode with an Agilent 1200 Series solvent manager, column manager, sample manager and PDA detector. The column for standard method was Purospher® STAR RP-18 endcapped 2 μm, Hibar® HR 50-2.1, the column temperature was 60° C., the flow rate was 0.8 mL/min, and injection volume was 0.5-5 μL. UV detection was in the range 210-400 nm. The mobile phase consisted of solvent A (water plus 0.05% formic acid) and solvent B (acetonitrile plus 0.05% formic acid) with different gradients for 2 different methods: 1) Starting with 98% solvent A changing to 100% solvent B over 1.8 minutes, maintained for 0.8 min; 2) Starting with 98% solvent A changing to 100% solvent B over 5.8 minutes, maintained for 0.3 minutes.

Preparative HPLC purifications were usually performed using a mass spectrometry directed system. Usually they were performed on a Waters Chromatography Workstation (MassLynx V4.1) configured with LC-MS System Consisting of: Waters ZQ™ 2000 (quad MS system with Electrospray Ionization), Waters 2545 Gradient Pump, Waters 2767 Injecto/Collector, Waters 2998 PDA Detector, the MS Conditions of: 100-1400 amu, Positive Electrospray, Collection Triggered by MS, and a Waters SUNFIRE® C-18 5 micron, 19 mm (id)×150 mm column. The mobile phases consisted of mixtures of acetonitrile (5-95%) in water containing 0.02% formic acid. Flow rates were maintained at 20 mL/min, the injection volume was 500 to 3000 μL, and the UV detection range was 210-400 nm. Mobile phase gradients were optimized for the individual compounds. The analytical system consisted of the same system with a Waters SUNFIRE® C-18 5 μm, 4.6×150 mm column, or a XSelect® CSH™ C-18 5 μm, 4.6×150 mm column. The mobile phases consisted of mixtures of acetonitrile (5-95%) in water containing 0.02% formic acid. Flow rates were maintained at 1.2 mL/min, the injection volume was 5 to 20 μL. Preparative HPLC were also performed on a Gilson system 233XL WITH 735 (Unipoint). The column was a Waters XBridge Prep C18 5 μm OBD, dimension 30×250 mm. The mobile phase consisted of mixture of acetonitrile/ammonium carbonate 0.02N (3-15% in 70 min or 3-30% in 50 min). Flow rates were maintained at 50 mL/min, the injection volume was 1000 μL, and the UV detection range was 260 nm.

Reactions performed using microwave irradiation were normally carried out using an Emrys Optimizer manufactured by Personal Chemistry, or an Initiator manufactured by Biotage. Concentration of solutions was carried out on a rotary evaporator in vacuo. Flash chromatography was usually performed using a Biotage® Flash Chromatography apparatus (Isolera) on silica gel (15-45 □□ 40-63 □, or spheric silica) in pre-packed cartridges of the size noted. ¹H NMR spectra were acquired at 400 MHz or 500 MHz spectrometers in CDCl₃ solutions unless otherwise noted. Chemical shifts were reported in parts per million (ppm). Tetramethylsilane (TMS) was used as internal reference in CDCl₃ solutions, and residual CH₃OH peak or TMS was used as internal reference in CD₃OD solutions. Coupling constants (J) were reported in hertz (Hz). Chiral analytical chromatography was performed on one of CHIRALPAK® AS, CHIRALPAK® AD, CHIRALCEL® OD, CHIRALCEL® IA, or CHIRALCEL® OJ columns (250×4.6 mm) (Daicel Chemical Industries, Ltd.) with noted percentage of either ethanol in hexane (% Et/Hex) or isopropanol in heptane (% IPA/Hep) as isocratic solvent systems. Chiral preparative chromatography was conducted on one of of CHIRALPAK AS, of CHIRALPAK AD, CHIRALCEL® OD, CHIRALCEL® IA, CHIRALCEL® OJ columns (20×250 mm) (Daicel Chemical Industries, Ltd.) with desired isocratic solvent systems identified on chiral analytical chromatography or by supercritical fluid (SFC) conditions.

### Preparation of Compounds

All nucleoside starting materials described in the following experimental procedures were prepared according to the protocols described in U.S. Pat. No. 7,339,053 or PCT publication WO2015/148746.

### Examples 1 and 2

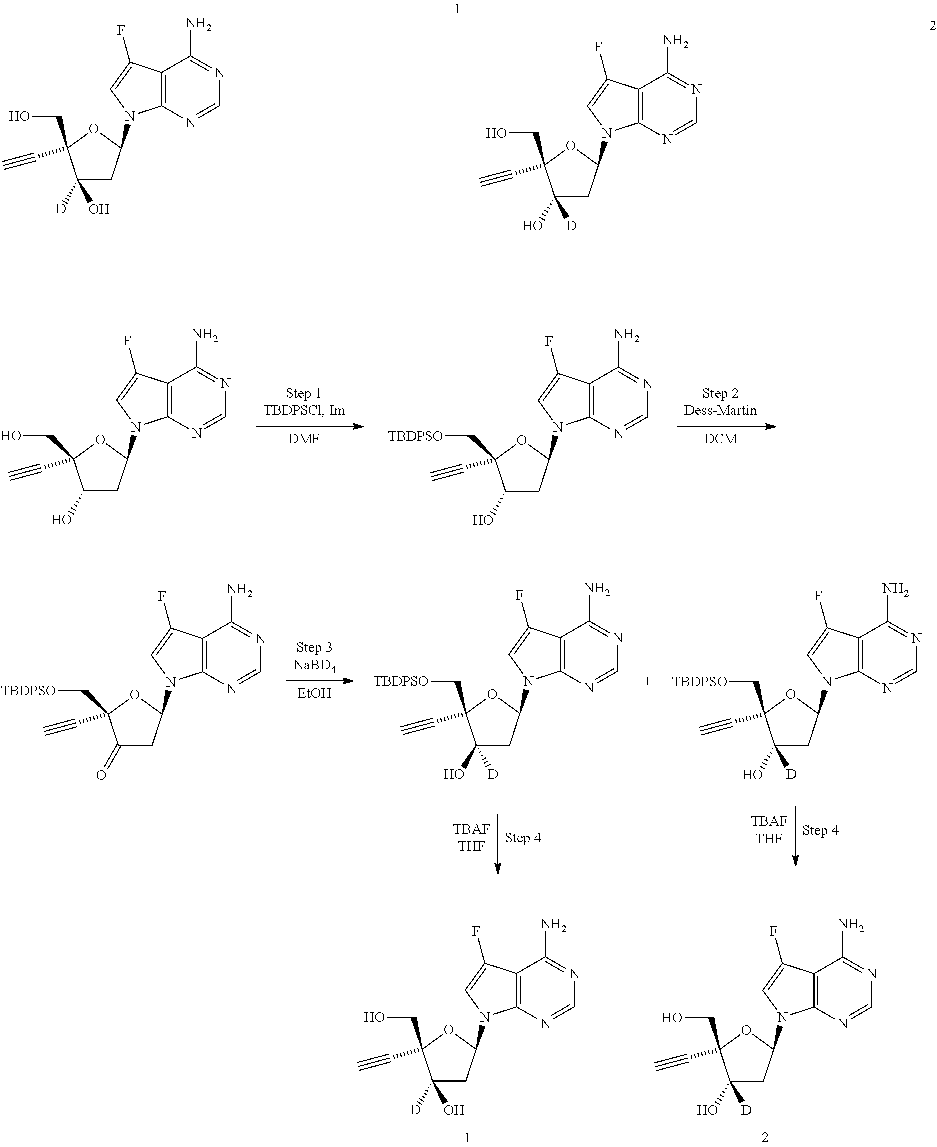

### Synthesis of (2R,3R,5R)-5-{4-amino-5-fluoro-7H-pyrrolo[2,3-d]pyrimidin-7-yl}-2-{[(tert-butyldiphenylsilyl)oxy]methyl}-2-ethynyl(3-²H)oxolan-3-ol (1) and (2R,3S,5R)-5-{4-amino-5-fluoro-7H-pyrrolo[2,3-d]pyrimidin-7-yl}-2-ethynyl-2-(hydroxymethyl)(3-²H)oxolan-3-ol (2)

(2R,3S,5R)-5-(4-amino-5-fluoro-7H-pyrrolo[2,3-d]pyrimidin-7-yl)-2-ethynyl-2-(hydroxymethyl)tetrahydrofuran-3-ol was prepared according to the protocols described in patent application PCT publication WO2015/148746.

Step 1: To a stirred solution of (2R,3S,5R)-5-(4-amino-5-fluoro-7H-pyrrolo[2,3-d]pyrimidin-7-yl)-2-ethynyl-2-(hydroxymethyl)tetrahydrofuran-3-ol (100 mg, 0.342 mmol) and imidazole (93 mg, 1.369 mmol) in dry DMF (1.5 mL) under argon atmosphere was injected a solution of TBDPSCl (0.114 mL, 0.445 mmol, in 0.1 mL DMF). The resulting mixture was stirred for 16 h at 25° C. The reaction progress was monitored by TLC. The reaction mixture was diluted with DCM (30 mL) and washed with water (15 mL) and brine (15 mL) successively. The organic layer was dried over anhydrous Na₂SO₄, filtered and the filtrate was concentrated under reduced pressure. The residue was purified by silica-gel column chromatography using 5% MeOH in DCM to give (2R,3S,5R)-5-(4-amino-5-fluoro-7H-pyrrolo[2,3-d]pyrimidin-7-yl)-2-(((tert-butyldiphenylsilyl)oxy)methyl)-2-ethynyltetrahydrofuran-3-ol (100 mg, 0.188 mmol, 55.1% yield) as a white foam. MS ESI calculated for C₂₉H₃₂FN₄O₃Si [M+H]⁺ 531.67, found 531.15.

Step 2: To a stirred solution of (2R,3S,5R)-5-(4-amino-5-fluoro-7H-pyrrolo[2,3-d]pyrimidin-7-yl)-2-(((tert-butyldiphenylsilyl)oxy)methyl)-2-ethynyltetrahydrofuran-3-ol (90 mg, 0.170 mmol) in DCM (4 mL) was added dess-martin periodinane (144 mg, 0.339 mmol) batchwise at 0° C. The resulting solution was stirred at ambient temperature for 1 h. The mixture was diluted with EA (2 mL), filtered and the filtrate was concentrated under reduced pressure to give (2R,5R)-5-(4-amino-5-fluoro-7H-pyrrol o[2,3-d]pyrimidin-7-yl)-2-(((tert-butyl diphenyl silyl)oxy)methyl)-2-ethynyl dihydrofuran-3(2H)-one (90 mg, 0.170 mmol, 100% yield) as a yellow solid, the solid was used to next step directly without further purification. MS ESI calculated for C₂₉H₃₀FN₄O₃Si [M+H]⁺ 529.65, found 529.15.

Step 3: To a stirred solution of ((2R,5R)-5-(4-amino-5-fluoro-7H-pyrrolo[2,3-d]pyrimidin-7-yl)-2-(((tert-butyldiphenylsilyl)oxy)methyl)-2-ethynyldihydrofuran-3(2H)-one (92 mg, 0.174 mmol) in ethanol (4 mL) was added sodium borodeuteride (14.57 mg, 0.348 mmol) batchwise at 0° C. The resulting solution was stirred at ambient temperature for 1 h. The pH value was adjusted to 7 with acetic acid and concentrated under reduced pressure. The residue was purified by silica-gel column chromatography, eluted with 5% MeOH in DCM to give (2R,3S,5R)-5-{4-amino-5-fluoro-7H-pyrrolo[2,3-d]pyrimidin-7-yl}-2-{[(tert-butyldiphenylsilyl)oxy]methyl}-2-ethynyl(3-²H)oxolan-3-ol (15 mg, 0.028 mmol, 16.21% yield) as a white foam, MS ESI calculated for C₂₉H₃₁DFN₄O₃Si [M+H]⁺ 532.67 [M+1]⁺, found 532.25. ¹H-NMR (300 MHz, Methanol-d₄, ppm): δ: 8.06 (s, 1H), 7.65-7.62 (m, 4H), 7.45-7.19 (m, 7H), 7.02 (d, J=2.2 Hz, 1H), 6.60-6.58 (m, 1H), 3.83 (s, 2H), 2.99 (s, 1H), 2.65-2.52 (m, 2H), 1.00 (s, 9H) and (2R,3R,5R)-5-{4-amino-5-fluoro-7H-pyrrolo[2,3-d]pyrimidin-7-yl}-2-{[(tert-butyldiphenylsilyl)oxy]methyl}-2-ethynyl(3-²H)oxolan-3-ol (20 mg, 0.038 mmol, 21.62% yield) as a white foam, MS ESI calculated for C₂₉H₃₁DFN₄O₃Si [M+H]⁺ 532.67, found 532.20. ¹H-NMR (300 MHz, Methanol-d₄, ppm): δ: 8.00 (s, 1H), 7.77-7.59 (m, 4H), 7.44-7.16 (m, 7H), 6.43-6.32 (m, 1H), 4.06 (d, J=10.1 Hz, 1H), 3.90 (d, J=10.0 Hz, 1H), 3.17-3.02 (m, 2H), 2.32-2.15 (m, 1H), 1.01 (s, 9H).

Step 4: TBAF (0.113 mL, 0.113 mmol) was added to a solution of (2R,3R,5R)-5-{4-amino-5-fluoro-7H-pyrrolo[2,3-d]pyrimidin-7-yl}-2-{[(tert-butyldiphenylsilyl)oxy]methyl}-2-ethynyl(3-²H)oxolan-3-ol (20 mg, 0.038 mmol) in THF (2 mL), the resulting solution was stirred at ambient temperature for 1 h, condensed under reduce pressure, the residue was purified with following conditions: column: XBridge C¹⁸ OBD Prep column, 100 Å, 5 μm, 19 mm×250 mm; Mobile Phase A:water (10 mmol/L NH₄HCO₃), Mobile Phase B: ACN; Flow rate: 20 mL/min; Gradient: 3% B to 30% B in 8 min; 254 nm; rt: 7.70 min to give (2R,3R,5R)-5-{4-amino-5-fluoro-7H-pyrrolo[2,3-d]pyrimidin-7-yl}-2-{[(tert-butyldiphenylsilyl)oxy]-methyl}-2-ethynyl(3-²H)oxolan-3-ol, 1, (2.4 mg, 8.18 μmol, 21.75% yield) as a white solid. MS ESI calculated for C₁₃H₁₃DFN₄O₃ [M+H]⁺ 294.27, found 294.10. ¹H-NMR (300 MHz, Methanol-d₄, ppm): δ: 8.04 (s, 1H), 7.17 (d, J=2.1 Hz, 1H), 6.57-6.55 (m, 1H), 3.75 (q, J=12.1 Hz, 2H), 3.03 (s, 1H), 2.62-2.41 (m, 2H).

TBAF (0.028 mL, 0.028 mmol) was added to a solution of (2R,3S,5R)-5-{4-amino-5-fluoro-7H-pyrrolo[2,3-d]pyrimidin-7-yl}-2-{[(tert-butyldiphenyl silyl)oxy]methyl}-2-ethynyl(3-²H)oxolan-3-ol (15 mg, 0.028 mmol) in THF (2 mL), the resulting solution was stirred at ambient temperature for 1 h, condensed under reduce pressure, the residue was purified with following conditions: column: XBridge C¹⁸ OBD Prep column, 100 Å, 5 μm, 19 mm×250 mm; Mobile Phase A:water (10 mM NH₄HCO₃), Mobile Phase B: ACN; Flow rate: 20 mL/min; Gradient: 3% B to 40% B in 8 min; 254 nm; rt: 8.02 min to give (2R,3S,5R)-5-{4-amino-5-fluoro-7H-pyrrolo[2,3-d]pyrimidin-7-yl}-2-ethynyl-2-(hydroxymethyl)(3-²H)oxolan-3-ol (2.5 mg, 8.52 μmol, 30.2% yield) as white solid. MS ESI calculated for C₁₃H₁₃DFN₄O₃ [M+H]⁺ 294.27, found 294.20. ¹H-NMR (300 MHz, Methanol-d₄, ppm): δ: 8.05 (s, 1H), 7.28 (d, J=2.1 Hz, 1H), 6.43-6.42 (m, 1H), 3.94-3.75 (m, 2H), 3.12-2.98 (m, 2H), 2.27 (dd, J=14.7, 3.4 Hz, 1H).

### Examples 3 and 4

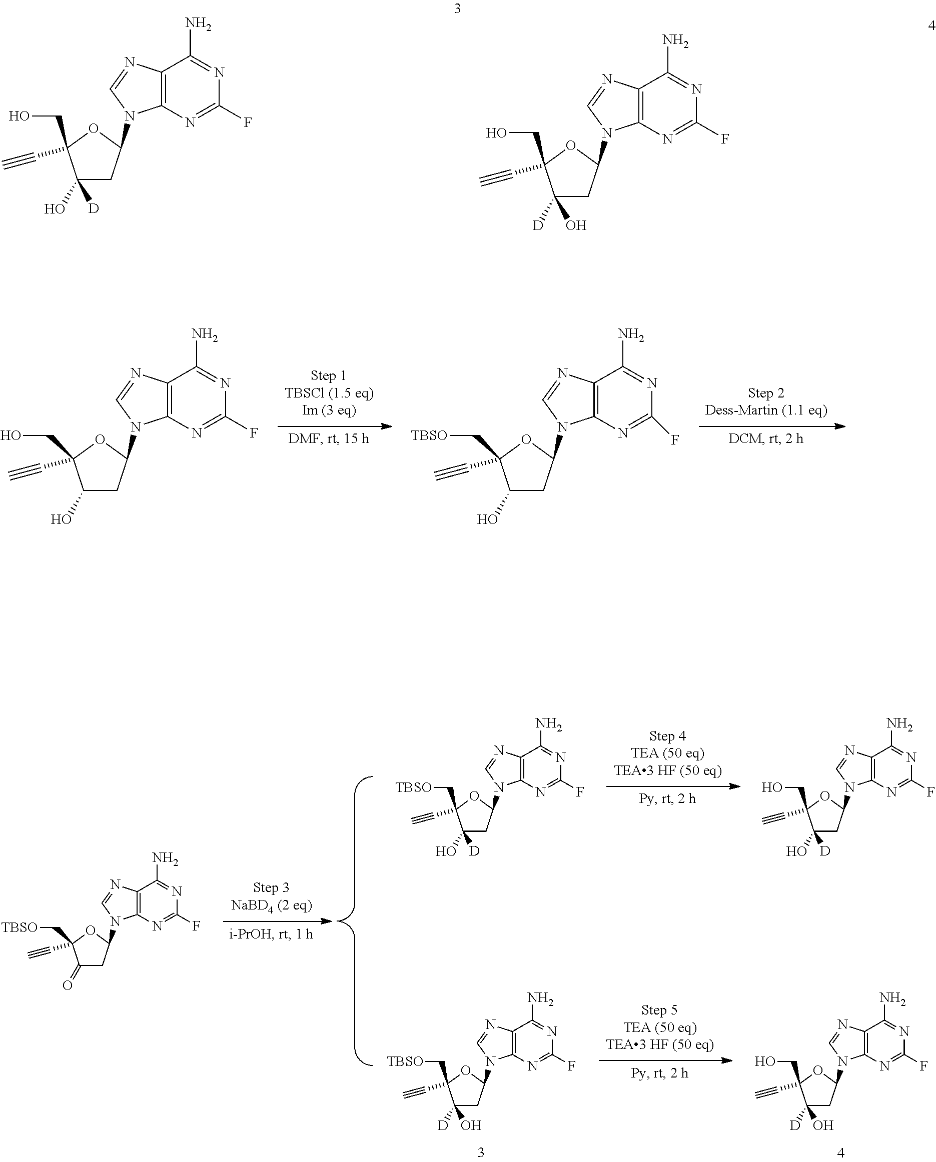

### Synthesis of (2R,3S,5R)-5-(6-amino-2-fluoro-9H-purin-9-yl)-2-ethynyl-2-(hydroxymethyl)(3-²H)oxolan-3-ol (3) and (2R,3R,5R)-5-(6-amino-2-fluoro-9H-purin-9-yl)-2-ethynyl-2-(hydroxymethyl)(3-²H)oxolan-3-ol (4)

(2R,3S,5R)-5-(6-amino-2-fluoro-9H-purin-9-yl)-2-ethynyl-2-(hydroxymethyl)tetrahydrofuran-3-ol was prepared according to the protocols described in U.S. Pat. No. 7,339,053.

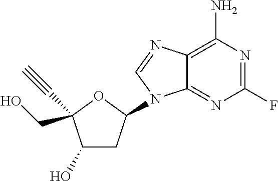 [SMILES: C#C[C@]1(CO)O[C@@H](n2cnc3c(N)nc(F)nc32)C[C@@H]1O]

Step 1: To a mixture of (2R,3S,5R)-5-(6-amino-2-fluoro-9H-purin-9-yl)-2-ethynyl-2-(hydroxymethyl)tetrahydrofuran-3-ol (100 mg, 0.341 mmol) and imidazole (69.6 mg, 1.023 mmol) in DMF (0.3 mL) was added TBSCl (77 mg, 0.512 mmol) under a argon atmosphere at 0° C. The reaction mixture was stirred for 15 h at 25° C. 65% desired product, 30% starting material was detected on LCMS. The reaction mixture was concentrated under reduced pressure, the residue was purified with silica gel column chromotagraphy, eluted with DCM/MeOH (10/1) to afford (2R,3S,5R)-5-(6-amino-2-fluoro-9H-purin-9-yl)-2-(((tert-butyldimethylsilyl)oxy)methyl)-2-ethynyltetrahydrofuran-3-ol (100 mg, 0.233 mmol, 68.4% yield) as an off-white silid. MS ESI calculated for C₁₈H₂₇FN₅O₃S [M+H]⁺ 408.51, found 408.25. ¹H-NMR: (400 MHz, d₆-DMSO, ppm) δ 8.21 (s, 1H), 7.81 (s, 2H), 6.20 (dd, J=7.7, 4.1 Hz, 1H), 5.59 (d, J=5.6 Hz, 1H), 4.61 (q, J=6.9 Hz, 1H), 3.81 (d, J=11.2 Hz, 1H), 3.70 (d, J=11.1 Hz, 1H), 3.52 (s, 1H), 2.77-2.76 (m, 1H), 2.48-2.38 (m, 1H), 0.78 (s, 9H), −0.08 (m, 6H).

Step 2: To a mixture of (2R,3S,5R)-5-(6-amino-2-fluoro-9H-purin-9-yl)-2-(((tert-butyldimethylsilyl)oxy)methyl)-2-ethynyltetrahydrofuran-3-ol (70 mg, 0.172 mmol) in DCM (2 mL) was added Dess-Martin Periodinane (80 mg, 0.189 mmol) under a argon atmosphere at 0° C. The resulting mixture was stirred for 2 h at 25° C. The major was desired product. The reaction was quenched with NaHCO₃ (20 mL), extracted with EA (100 mL), the organic layer was washed with Na₂S₂O₃ (sat., 20 mL), brine (sat., 20 mL), and dried with Na₂SO₄, evaporated under reduced pressure. The crude obtained was used directly to next step without further purification. MS ESI calculated for C₁₈H₂₅FN₅O3Si [M+H]⁺ 406.50, found 406.20.

Step 3: To a mixture of (2R,5R)-5-(6-amino-2-fluoro-9H-purin-9-yl)-2-(((tert-butyldimethylsilyl)oxy)methyl)-2-ethynyldihydrofuran-3(2H)-one (60 mg, 0.148 mmol) in 2-Propanol (1 mL) was added sodium borodeuteride (12.39 mg, 0.296 mmol) under a argon atmosphere at 0° C. The resulting mixture was stirred for 1 h at 25° C. The major was desired product. The reaction was quenched with HOAc (1 d) at 0° C. and concentrated under reduced pressure. The residue was purified with silica gel column chromatography, eluted with DCM/MeOH (11/1) to afford (2R,3S,5R)-5-(6-amino-2-fluoro-9H-purin-9-yl)-2-{[(tert-butyldimethylsilyl)oxy]methyl}-2-ethynyl(3-²H)oxolan-3-ol (20 mg, 0.047 mmol, 31.4% yield) as an off-white solid, MS ESI calculated for C₁₈H₂₆DFN₅O₃Si [M+H]⁺ 409.52, found 409.15; and (2R,3R,5R)-5-(6-amino-2-fluoro-9H-purin-9-yl)-2-{[(tert-butyldimethylsilyl)oxy]methyl}-2-ethynyl(3-²H)oxolan-3-ol (15 mg, 0.035 mmol, 23.57% yield) as an off-white solid, MS ESI calculated for C₁₈H₂₆DFN5O₃Si [M+H]⁺ 409.52, found 409.07.

Step 4: To a mixture of (2R,3S,5R)-5-(6-amino-2-fluoro-9H-purin-9-yl)-2-{[(tert-butyldimethylsilyl)oxy]methyl}-2-ethynyl(3-²H)oxolan-3-ol (15 mg, 0.037 mmol) in Py (0.7 mL) were added TEA (0.256 mL, 1.836 mmol) and triethylamine trihydrofluoride (296 mg, 1.836 mmol) under a argon atmosphere at 20° C. The resulting mixture was stirred for 2 h at 25° C. The major was desired product. The reaction was concentrated under reduced pressure. The residue was purified with Prep-TLC, eluted with DCM/MeOH (11/1) to afford a crude product. The crude product was purified with prep-HPLC, condition: Column: XBridge Prep Phenyl OBD Column 19×150 mm 5 um 13 nm; Mobile Phase A:Water (10 mM NH₄HCO3), Mobile Phase B: ACN; Flow rate: 20 mL/min; Gradient: 9% B to 14% B in 11 min; 210 nm; Rt: 7.70 min to afford (2R,3S,5R)-5-(6-amino-2-fluoro-9H-purin-9-yl)-2-ethynyl-2-(hydroxymethyl)(3-²H)oxolan-3-ol, 3, (5 mg, 0.017 mmol, 45.4% yield) as an off-white solid. MS ESI calculated for C₁₂H₁₂DFN₅O₃ [M+H]⁺ 295.26, found 295.25. ¹H-NMR: (400 MHz, d₆-DMSO, ppm) δ 8.28 (s, 1H), 7.83 (s, 2H), 6.22 (dd, J=7.3, 5.0 Hz, 1H), 5.58-5.50 (m, 1H), 5.27-5.26 (m, 1H), 3.63-3.62 (m, 1H), 3.53-3.52 (m, 1H), 3.49 (s, 1H), 2.68-2.67 (m, 1H), 2.46-2.35 (m, 1H). F-NMR: (400 MHz, d₆-DMSO, ppm) δ −51.984 (s, 1F).

Step 5: To a mixture of (2R,3R,5R)-5-(6-amino-2-fluoro-9H-purin-9-yl)-2-{[(tert-butyldimethylsilyl)oxy]methyl}-2-ethynyl(3-²H)oxolan-3-ol (15 mg, 0.037 mmol) in Py (0.3 mL) were added TEA (0.256 mL, 1.836 mmol) and triethylamine trihydrofluoride (296 mg, 1.836 mmol) under a argon atmosphere at 20° C. The resulting mixture was stirred for 2 h at 25° C. The major was desired product. The reaction was concentrated under reduced pressure. The residue was purified with Prep-TLC, eluted with DCM/MeOH (11/1) to afford a crude product. The crude product was purified with Prep-HPLC, Column: XBridge Prep Phenyl OBD Column 19×150 mm 5 um 13 nm; Mobile Phase A:Water (10 mM aq. NH₄HCO₃), Mobile Phase B: ACN; Flow rate: 20 mL/min; Gradient: 9% B to 14% B in 11 min; 210 nm; Rt: 5.43 min to afford (2R,3R,5R)-5-(6-amino-2-fluoro-9H-purin-9-yl)-2-ethynyl-2-(hydroxymethyl)(3-²H)oxolan-3-ol, 4, (5 mg, 0.017 mmol, 45.8% yield) as an off-white solid. MS ESI calculated for C₁₂H₁₂DFN₅O₃ [M+H]⁺ 295.26, found 295.20. ¹H-NMR: (400 MHz, d₆-DMSO, ppm) δ 8.29 (s, 1H), 7.88 (s, 2H), 6.22 (dd, J=8.4, 3.2 Hz, 1H), 5.98 (s, 1H), 4.97 (t, J=6.2 Hz, 1H), 3.67-3.66 (m, 2H), 3.55 (s, 1H), 2.94-2.93 (m, 1H), 2.32-2.30 (m, 1H). F-NMR: (400 MHz, d₆-DMSO, ppm) 6-52.263 (s, 1F).

### Examples 5 and 6

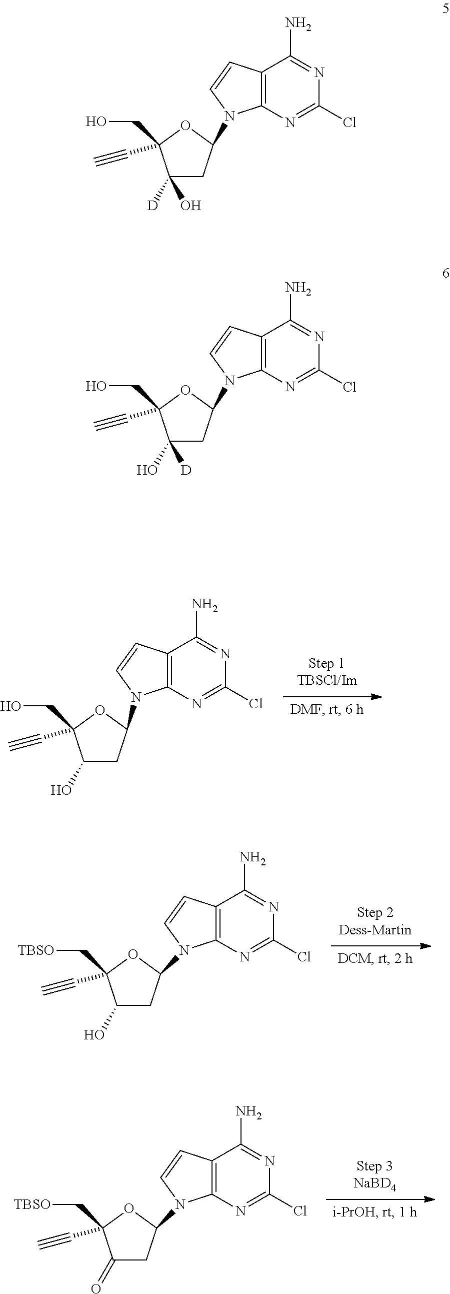

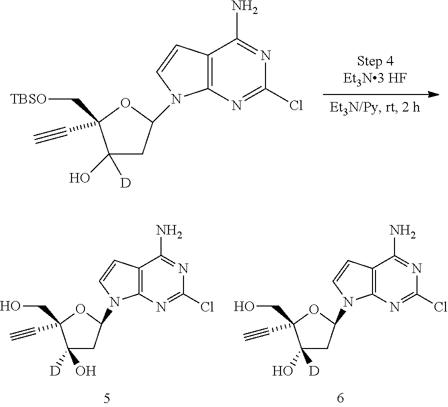 [SMILES: [2H]C1(O)CC(n2ccc3c(N)nc(Cl)nc32)O[C@]1(C#C)CO[Si](C)(C)C(C)(C)C ; "Et3N/Py, rt, 2 h" ; [2H][C@@]1(O)C[C@H](n2ccc3c(N)nc(Cl)nc32)O[C@]1(C#C)CO ; [2H][C@]1(O)C[C@H](n2ccc3c(N)nc(Cl)nc32)O[C@]1(C#C)CO]

### Synthesis of (2R,3R,5R)-5-(4-amino-2-chloro-7H-pyrrolo[2,3-d]pyrimidin-7-yl)-2-ethynyl(3-²H)-2-(hydroxymethyl)-tetrahydrofuran-3-ol (5) and (2R,3S,5R)-5-(4-amino-2-chloro-7H-pyrrolo[2,3-d]pyrimidin-7-yl)-2-ethynyl(3-²H)-2-(hydroxymethyl)-tetrahydrofuran-3-ol (6)

(2R,3S,5R)-5-(4-amino-2-chloro-7H-pyrrolo[2,3-d]pyrimidin-7-yl)-2-ethynyl-2-(hydroxymethyl)-tetrahydrofuran-3-ol was prepared according to the protocols described in PCT publication WO2015/148746.

Step 1: To a mixture of (2R,3S,5R)-5-(4-amino-2-chloro-7H-pyrrolo[2,3-d]pyrimidin-7-yl)-2-ethynyl-2-(hydroxymethyl)-tetrahydrofuran-3-ol (100 mg, 0.32 mmol) and imidazole (66 mg, 0.96 mmol) in DMF (0.3 mL) was added TBSCl (66 mg, 0.39 mmol) under a argon atmosphere at 0° C. The reaction mixture was stirred for 6 h at 25° C. The major was desired product, trace starting material was detected on LCMS. The reaction mixture was concentrated under reduced pressure, the residue was purified with silica gel column chromatography, eluted with DCM/MeOH (10/1) to afford (2R,3S,5R)-5-(4-amino-2-chloro-7H-pyrrolo[2,3-d]pyrimidin-7-yl)-2-((tert-butyldimethylsilyloxy)methyl)-2-ethynyl-tetrahydrofuran-3-ol (120 mg, 0.28 mmol, 87.5% yield) as an off-white solid. MS ESI calculated for C₁₉H₂₈ClN₄O₃Si [M+H]⁺ 423.15, found 423.15. ¹H-NMR: (400 MHz, DMSO-d₆, ppm): δ 7.55 (s, 2H), 7.28 (d, J=3.7 Hz, 1H), 6.60 (d, J=3.6 Hz, 1H), 6.39 (dd, J=7.4, 5.3 Hz, 1H), 5.59 (d, J=5.6 Hz, 1H), 4.50 (q, J=6.8 Hz, 1H), 3.80 (d, J=11.0 Hz, 1H), 3.72 (d, J=11.1 Hz, 1H), 3.53 (s, 1H), 2.92-2.90 (m, 1H), 2.40-2.38 (m, 1H), 0.87 (s, 9H), 0.04 (s, 6H).

Step 2: To a mixture of (2R,3S,5R)-5-(4-amino-2-chloro-7H-pyrrolo[2,3-d]pyrimidin-7-yl)-2-((tert-butyldimethylsilyloxy)methyl)-2-ethynyl-tetrahydrofuran-3-ol (120 mg, 0.28 mmol) in DCM (2 mL) was added dess-martinperiodinane (237 mg, 0.56 mmol) under an argon atmosphere at 0° C. The resulting mixture was stirred for 2 h at 25° C. The major was desired product. The reaction was quenched with NaHCO₃ (20 mL), extracted with EA (100 mL), the organic layer was washed with Na₂S₂O₃ (sat., 20 mL), brine (sat., 20 mL), and dried with Na₂SO₄, evaporated under reduced pressure. The crude obtained was used directly to next step without further purification. MS ESI calculated for C₁₉H₂₆ClN₄O₃Si [M+H]⁺ 421.14, found 421.20.

Step 3: To a mixture of (2R,5R)-5-(4-amino-2-chloro-7H-pyrrolo[2,3-d]pyrimidin-7-yl)-2-((tert-butyldimethylsilyloxy)methyl)-2-ethynyl-dihydrofuran-3(2H)-one (190 mg, 0.45 mmol) in i-propanol (6 mL) was added sodium borodeuteride (38.4 mg, 0.9 mmol) under an argon atmosphere at 0° C. The resulting mixture was stirred for 1 h at 25° C. The major was desired product. The reaction was quenched with HOAc (1 d) at 0° C. and concentrated under reduced pressure. The residue was purified with silica gel column chromatography, eluted with DCM/MeOH (11/1) to afford (2R,5R)-5-(4-amino-2-chloro-7H-pyrrolo[2,3-d]pyrimidin-7-yl)-2-((tert-butyldimethylsilyloxy)methyl)-2-ethynyl(3-²H₁)-tetrahydrofuran-3-ol (48 mg, 0.113 mmol, 25.1% yield) as an off-white solid. MS ESI calculated for C₁₉H₂₇DClN₄O₃Si [M+H]⁺ 424.16, found 424.30. ¹H NMR (400 MHz, DMSO-d₆) δ 7.60 (s, 2H), 7.48-7.42 (m, 1H), 6.61 (d, J=3.6 Hz, 1H), 6.37 (dd, J=8.7, 3.7 Hz, 1H), 6.08 (s, 1H), 3.92 (d, J=10.3 Hz, 1H), 3.78 (d, J=10.3 Hz, 1H), 3.56 (s, 1H), 2.95 (dd, J=14.4, 8.8 Hz, 1H), 2.20-2.11 (m, 1H), 0.89 (d, J=6.5 Hz, 9H), 0.11-0.03 (m, 6H).

Step 4: To a mixture of (2R,3R,5R)-5-(4-amino-2-chloro-7H-pyrrolo[2,3-d]pyrimidin-7-yl)-2-((tert-butyldimethylsilyloxy)methyl)-2-ethynyl(3-²H₁)-tetrahydrofuran-3-ol (18 mg, 0.04 mmol) in Py (0.20 mL) were added Et₃N (202.2 mg, 2.0 mmol) and triethylamine trihydrofluoride (322.4 mg, 2.0 mmol) under an argon atmosphere at 20° C. The resulting mixture was stirred for 2 h at 25° C. The major was desired product. The reaction mixture was concentrated under reduced pressure. The residue was purified by silica-gel column chromatography, eluting with 5% MeOH in DCM to give a crude product. The crude product was purified by Prep-HPLC: Column: XBridge Prep Phenyl OBD Column 19×150 mm 5 um 13 nm; Mobile Phase A: Water (10 mM NH₄HCO₃), Mobile Phase B: ACN; Flow rate: 20 mL/min; Gradient: 6% B to 34% B in 9 min; 210 nm; Rt: 7.68 min to afford (2R,3R,5R)-5-(4-amino-2-chloro-7H-pyrrolo[2,3-d]pyrimidin-7-yl)-2-ethynyl (3-²H₁)-2-(hydroxymethyl)-tetrahydrofuran-3-ol, 5, (8 mg, 0.02 mmol, 64.7% yield) as an off-white solid, MS ESI calculated for C₁₃H₁₃DClN4O₃ [M+H]⁺ 310.07, found 310.20. ¹H-NMR: (400 MHz, DMSO-d₆, ppm): δ7.59 (s, 2H), 7.50-7.43 (m, 1H), 6.60 (d, J=3.6 Hz, 1H), 6.38 (dd, J=8.7, 3.7 Hz, 1H), 5.99 (s, 1H), 4.93 (t, J=6.2 Hz, 1H), 3.72 (dd, J=11.1, 6.3 Hz, 1H), 3.62 (dd, J=11.1, 6.2 Hz, 1H), 3.54 (s, 1H), 2.95 (dd, J=14.5, 8.7 Hz, 1H), 2.20-2.06 (m, 1H) And (2R,3S,5R)-5-(4-amino-2-chloro-7H-pyrrolo[2,3-d]pyrimidin-7-yl)-2-ethynyl(3-²H₁)-2-(hydroxymethyl)-tetrahydrofuran-3-ol, 6, (10 mg, 0.03 mmol, 46.23% yield) as an off-white solid, MS ESI calculated for C₁₃H₁₃DClN4O₃ [M+H]⁺ 310.07, found 310.20. ¹H-NMR: (400 MHz, DMSO-d₆, ppm): δ 7.56 (s, 2H), 7.37-7.30 (m, 1H), 6.61 (d, J=3.7 Hz, 1H), 6.42 (dd, J=7.1, 5.8 Hz, 1H), 5.52 (s, 1H), 5.27 (t, J=6.1 Hz, 1H), 3.67-3.46 (m, 1H), 3.37 (s, 1H), 3.32 (s, 1H), 2.48 (d, J=5.8 Hz, 1H), 2.37-2.36 (m, 1H).

### Example 7

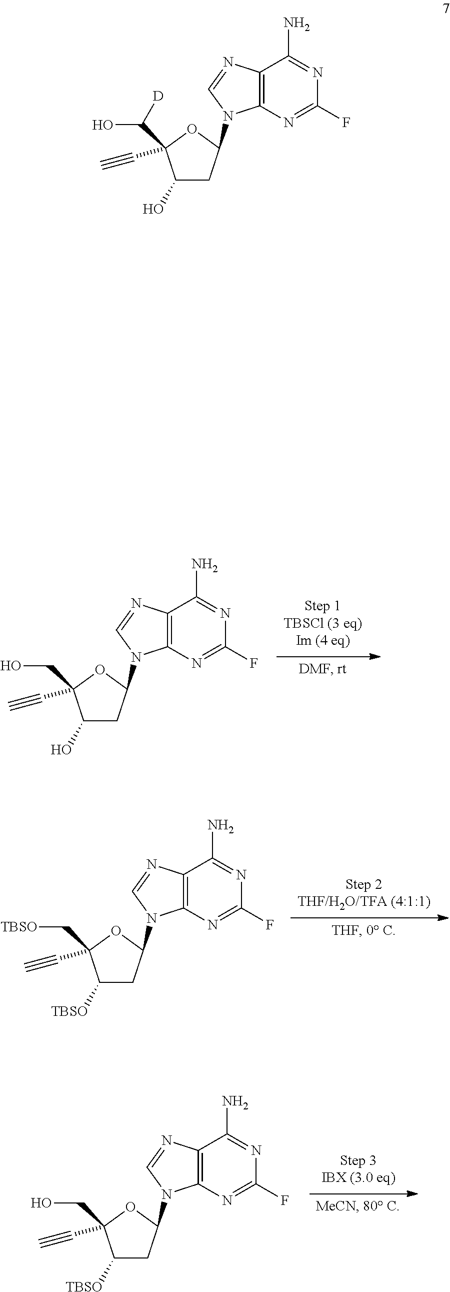

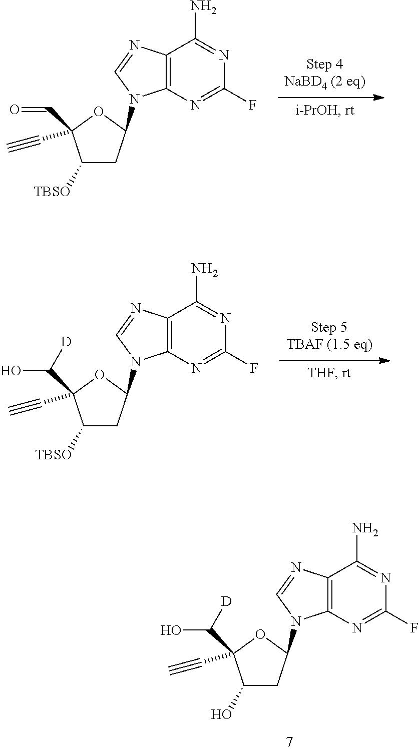 [SMILES: C#C[C@]1(C=O)O[C@@H](n2cnc3c(N)nc(F)nc32)C[C@@H]1O[Si](C)(C)C(C)(C)C ; "i-PrOH, rt" ; [2H]C(O)[C@@]1(C#C)O[C@@H](n2cnc3c(N)nc(F)nc32)C[C@@H]1O[Si](C)(C)C(C)(C)C ; "THF, rt" ; [2H]C(O)[C@@]1(C#C)O[C@@H](n2cnc3c(N)nc(F)nc32)C[C@@H]1O]

### Synthesis of [(2R,3S,5R)-5-(6-amino-2-fluoro-9H-purin-9-yl)-3-oxhydryl-2-ethynyloxolan-2-yl](²H)-methanol (7)

(2R,3S,5R)-5-(6-amino-2-fluoro-9H-purin-9-yl)-2-ethynyl-2-(hydroxymethyl)tetrahydrofuran-3-ol was prepared according to the protocols described in U.S. Pat. No. 7,339,053.

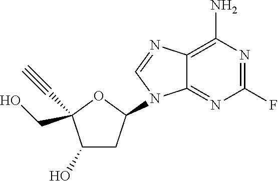 [SMILES: C#C[C@]1(CO)O[C@@H](n2cnc3c(N)nc(F)nc32)C[C@@H]1O]

Step 1: To a stirred solution of (2R,3S,5R)-5-(6-amino-2-fluoro-9H-purin-9-yl)-2-ethynyl-2-(hydroxymethyl)tetrahydrofuran-3-ol (120 mg, 0.409 mmol) and imidazole (111 mg, 1.637 mmol) in dry DMF (0.2 mL) under argon atmosphere was injected a solution of tert-butylchlorodimethylsilane (185 mg, 1.228 mmol) in 0.1 mL DMF. The resulting mixture was stirred for 3 h at 25° C. The reaction progress was monitored by TLC. The reaction mixture was diluted with DCM (20 mL) and washed with water (10 mL) and brine (10 mL) successively. The organic layer was dried over anhydrous Na₂SO₄, filtered and the filtrate was concentrated under reduced pressure. The residue was purified by silica gel column chromatography using 5% methanol in dichloromethane to give 9-((2R,4S,5R)-4-((tert-butyldimethylsilyl)oxy)-5-(((tert-butyldimethylsilyl)oxy)methyl)-5-ethynyltetrahydrofuran-2-yl)-2-fluoro-9H-purin-6-amine (150 mg, 0.287 mmol, 70.3% yield) as an off-white foam. MS ESI calculated for C₂₄H₄₁FN₅O₃Si₂ [M+H]⁺ 522.27, found 522.20. ¹H-NMR: (300 MHz, DMSO-d₆, ppm): δ 8.20 (s, 1H), 7.78 (s, 2H), 6.21 (dd, J=7.7, 4.1 Hz, 1H), 4.94-4.83 (m, 1H), 3.76 (d, J=11.2 Hz, 1H), 3.60 (d, J=11.2 Hz, 1H), 3.50 (s, 1H), 2.90-2.88 (m, 1H), 2.45-2.32 (m, 1H), 0.88 (s, 9H), 0.72 (s, 9H), 0.11 (s, 6H), −0.07 (s, 3H), −0.19 (s, 3H).

Step 2: THF/H₂O/TFA (4/1/1) (3 mL) was added dropwise to a solution of 9-((2R,4S,5R)-4-((tert-butyldimethylsilyl)oxy)-5-(((tert-butyldimethylsilyl)oxy)methyl)-5-ethynyltetrahydrofuran-2-Y¹)-2-fluoro-9H-purin-6-amine (150 mg, 0.287 mmol) at 0° C., the resulting solution was allowed to react at the same temperature for 16 h, coevaporated with toluene for three times, the residue was purified by a silica-gel column chromatography, eluting with 5% MeOH in DCM to give ((2R,3S,5R)-5-(6-amino-2-fluoro-9H-purin-9-yl)-3-((tert-butyldimethylsilyl)oxy)-2-ethynyltetrahydrofuran-2-yl)methanol (70 mg, 0.172 mmol, 59.8% yield) as an off-white solid. MS ESI calculated for C₁₈H₂₇FN₅O₃Si [M+H]⁺ 408.18, found 408.30. ¹H-NMR: (400 MHz, DMSO-d₆, ppm): δ 8.32 (s, 1H), 7.87 (s, 2H), 6.27 (dd, J=7.1, 5.6 Hz, 1H), 5.35 (dd, J=6.9, 5.5 Hz, 1H), 4.78 (t, J=6.2 Hz, 1H), 3.67 (dd, J=12.0, 5.5 Hz, 1H), 3.57-3.47 (m, 2H), 2.84-2.82 (m, 1H), 2.40-2.38 (m, 1H), 0.92 (s, 9H), 0.14 (d, J=3.2 Hz, 6H).

Step 3: IBX (144 mg, 0.515 mmol) was added to a solution of ((2R,3S,5R)-5-(6-amino-2-fluoro-9H-purin-9-yl)-3-((tert-butyldimethylsilyl)oxy)-2-ethynyltetrahydrofuran-2-yl)methanol (70 mg, 0.172 mmol) in acetonitrile (5 mL). The resulting mixture was allowed to react at 80° C. for 30 min, cooled to ambient temperature, filtered, the filtrate was condensed under reduced pressure to give (2S,3S,5R)-5-(6-amino-2-fluoro-9H-purin-9-yl)-3-((tert-butyldimethylsilyl)oxy)-2-ethynyltetrahydrofuran-2-carbaldehyde (70 mg, 0.173 mmol, 100% yield) as a crude yellow solid, the solid was used to next step directly without further purification. MS ESI calculated for C₁₈H₂₅FN₅O₃Si [M+H]⁺ 406.16, found 406.30.

Step 4: To a stirred solution of (2S,3S,5R)-5-(6-amino-2-fluoro-9H-purin-9-yl)-3-((tert-butyldimethylsilyl)oxy)-2-ethynyltetrahydrofuran-2-carbaldehyde (70 mg, 0.173 mmol) in 2-propanol (4 mL) was added sodium borodeuteride (14.45 mg, 0.345 mmol) batchwise at 0° C. The resulting solution was stirred at rt for 1 h. The pH value of the system was adjusted to 7 with acetic acid and concentrated under reduced pressure, the residue was purified by silica-gel column chromatography, eluting with 5% MeOH in DCM to give [(2R,3S,5R)-5-(6-amino-2-fluoro-9H-purin-9-yl)-3-[(tert-butyldimethylsilyl)oxy]-2-ethynyloxolan-2-yl](²H₁)methanol (40 mg, 0.098 mmol, 56.7% yield) as a white solid. MS ESI calculated for C₁₈H₂₆DFN₅O₃Si [M+H]⁺ 409.19, found 409.20. ¹H-NMR: (300 MHz, DMSO-d₆, ppm): δ 8.28 (s, 1H), 7.82 (s, 2H), 6.22 (dd, J=7.0, 5.6 Hz, 1H), 5.32-5.23 (m, 1H), 4.73 (t, J=6.2 Hz, 1H), 3.61-3.46 (m, 2H), 2.80-2.79 (m, 1H), 2.36-2.35 (m, 1H), 0.88 (s, 9H), 0.10 (d, J=2.3 Hz, 6H).

Step 5: To a solution of [(2R,3S,5R)-5-(6-amino-2-fluoro-9H-purin-9-yl)-3-[(tert-butyldimethylsilyl)oxy]-2-ethynyloxolan-2-yl](²H₁)methanol (40 mg, 0.098 mmol) in THF (1 mL) was added TBAF (0.15 mL, 0.15 mmol) at 0° C. under an argon atmosphere. The mixture was stirred at rt for 2 h. The mixture was concentrated under the vacuum. The residue was purified by silica-gel column chromatography, eluting with 5% MeOH in DCM to give a crude product. The crude product was purified with Prep-HPLC with the following conditions: Column: XBridge Prep C18 OBD Column, 5 um, 19*150 mm; Mobile Phase A: Water (10 mM NH₄HCO₃), Mobile Phase B: ACN; Flow rate: 20 mL/min; Gradient: 9% B to 35% B in 9 min; 254/210 nm; Rt: 6.3 min to afford [(2R,3S,5R)-5-(6-amino-2-fluoro-9H-purin-9-yl)-3-oxhydryl-2-ethynyloxolan-2-yl](²H₁)methanol, 7, (20 mg, 0.068 mmol, 69.42% yield) as a white solid. MS ESI calculated for C₁₂H₁₂DFN₅O₃ [M+H]⁺ 295.10, found 295.00. ¹H-NMR: (400 MHz, DMSO-d₆, ppm): δ 8.28 (s, 1H), 7.83 (s, 2H), 6.22 (dd, J=7.2, 5.0 Hz, 1H), 5.55 (d, J=5.4 Hz, 1H), 5.26 (d, J=6.0 Hz, 1H), 4.55 (q, J=6.6 Hz, 1H), 3.61 (d, J=5.5 Hz, 0.6H), 3.51 (d, J=14.4 Hz, 1.4H), 2.68-2.65 (m, 1H), 2.40-2.38 (m, 1H). F-NMR: (376 MHz, DMSO-d₆, ppm): δ −51.986 (s, 1F).

### Example 8

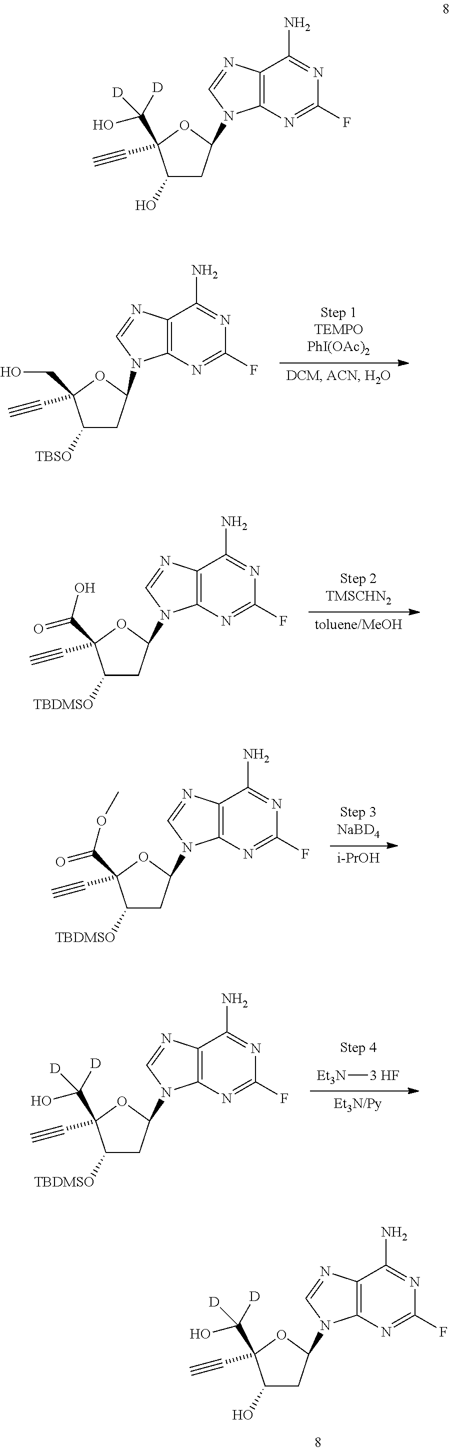

### Synthesis of [(2R,3S,5R)-5-(6-amino-2-fluoro-9H-purin-9-yl)-3-oxhydryl-2-ethynyloxolan-2-yl](²H₂)methanol (8)

((2R,3S,5R)-5-(6-amino-2-fluoro-9H-purin-9-yl)-3-(tert-butyldimethylsilyloxy)-2-ethynyl-tetrahydrofuran-2-yl) methanol was prepared according to the protocol described in EXAMPLE 7.

Step 1: To a solution of ((2R,3S,5R)-5-(6-amino-2-fluoro-9H-purin-9-yl)-3-(tert-butyldimethylsilyloxy)-2-ethynyl-tetrahydrofuran-2-yl) methanol (150 mg, 0.37 mmol) in DCM (5 mL) were added TEMPO (11.6 mg, 0.073 mmol) and PhI(OAc)₂ (266.3 mg, 0.442 mmol) at 25° C. After the mixture was stirred for 2 h, a mixture of MeCN (0.050 mL)-water (0.050 mL) was added to the reaction mixture, and the mixture was stirred for 15 h. The reaction progress was monitored by LCMS, the solid was filtered and washed with ether/acetone=2/1 (3×5 mL) to afford (2S,3S,5R)-5-(6-amino-2-fluoro-9H-purin-9-yl)-3-(tert-butyl dimethyl silyl oxy)-2-ethynyl-tetrahydrofuran-2-carboxylic acid (90 mg, 0.213 mmol, 57.6% yield) as a white solid. MS ESI calculated for C₁₈H₂₅FN₅O₄Si [M+H]⁺ 422.16, found 422.20. ¹H-NMR: (400 MHz, DMSO-d₆, ppm): δ 13.63 (s, 1H), 8.32 (s, 1H), 7.88 (s, 2H), 6.36 (t, J=6.8 Hz, 1H), 4.96 (t, J=4.7 Hz, 1H), 3.71 (s, 1H), 2.87-2.85 (m, 1H), 2.54-2.53 (m, 1H), 0.94 (s, 9H), 0.17 (s, 6H).

Step 2: To a solution of (2S,3R,5R)-3-(tert-butyldiphenylsilanyloxy)-5-(5-methyl-2,4-di-oxo-3,4-dihydro-2H-pyrimidin-1-yl)tetrahydrofuran-2-carbox-ylicacid (74 mg, 0.175 mmol) in toluene (2 mL)-MeOH (1.6 mL) was added (trimethylsilyl)diazomethane (2M in hexane, 0.17 mL, 3.4 mmol) at room temperature, and the mixture was stirred for 10 h. After removal of the solvent, the residue was purified by flash chromatography (PE/EA=1/1) to give (2S,3R,5R)-3-(tert-butyldiphenylsilanyloxy)-5-(5-meth-yl-2,4-dioxo-3,4-dihydro-2H-pyrimidin-1-yl)tetrahydrofuran-2-carboxylicacidmethylester (70 mg, 0.16 mmol, 85.7% yield) as a white solid. MS ESI calculated for C₁₉H₂₇FN₅O₄Si [M+H]⁺ 436.17, found 436.30. ¹H-NMR: (400 MHz, DMSO-d₆, ppm): δ 8.31 (s, 1H), 7.90 (s, 2H), 6.35-6.33 (m, 1H), 5.03 (s, 1H), 3.81 (s, 1H), 3.73 (s, 3H), 2.91-2.88 (m, 1H), 2.48 (s, 1H), 0.94 (s, 9H), 0.17 (s, 6H).

Step 3: To a stirred solution of (2S,3S,5R)-methyl 5-(6-amino-2-fluoro-9H-purin-9-yl)-3-(tert-butyldimethylsilyloxy)-2-ethynyl-tetrahydrofuran-2-carboxylate (70 mg, 0.173 mmol) in 2-propanol (4 mL) was added sodium borodeuteride (14.45 mg, 0.345 mmol) batchwise at 0° C. The resulting solution was stirred at rt for 1 h. The pH value of the reaction system was adjusted to 7 with acetic acid and concentrated under reduced pressure, the residue was purified by silica-gel column chromatography, eluting with 5% MeOH in DCM to give (40 mg, 0.098 mmol, 56.7% yield) as a white solid. MS ESI calculated for C₁₈H₂₅D₂FN₅O₃Si [M+H]⁺ 410.19, found 410.20. ¹H-NMR (400 MHz, DMSO-d₆, ppm): δ 8.30 (s, 1H), 7.91 (s, 2H), 6.38 (t, J=6.6 Hz, 1H), 5.08 (t, J=4.9 Hz, 1H), 4.92-4.90 (m, 1H), 3.77 (s, 1H), 2.98-2.96 (m, 1H), 2.54-2.51 (m, 1H), 0.94 (s, 9H), 0.17 (s, 6H).

Step 4: Into a 25 mL round-bottom flask was placed [(2R,3S,5R)-5-(6-amino-2-fluoro-9H-purin-9-yl)-3-[(tert-butyldimethylsilyl)oxy]-2-ethynyloxolan-2-yl](²H₁)methanol (40 mg, 0.098 mmol) under the argon atmosphere. To this was added a mixture of pyridine (0.5 mL) and Et₃N (0.5 mL). Then Et₃N.3HF (1 mL) was added at 0° C. The mixture was stirred at rt for 2 h. The mixture was concentrated under the vacuum, the residue was purified by silica-gel column chromatography, eluting with 5% MeOH in DCM to afford a crude product. The crude product was purified with Prep-HPLC with the following conditions: Column: XBridge Prep Phenyl OBD Column 19×150 mm 5 um 13 nm; Mobile Phase A:Water (10 mM NH₄HCO₃), Mobile Phase B: ACN; Flow rate: 20 mL/min; Gradient: 2% B to 26% B in 9 min; 254 nm; Rt: 6.00 min to give [(2R,3S,5R)-5-(6-amino-2-fluoro-9H-purin-9-yl)-3-oxhydryl-2-ethynyloxolan-2-yl](²H₂)methanol, 8, (20 mg, 0.068 mmol, 69.42% yield) as a white solid. MS ESI calculated for C₁₂H₁₁D₂FN₅O₃ [M+H]⁺ 296.10, found 296.20. ¹H-NMR: (400 MHz, DMSO-d₆, ppm): δ 8.28 (s, 1H), 7.83 (s, 2H), 6.22 (dd, J=7.3, 5.0 Hz, 1H), 5.54 (d, J=5.5 Hz, 1H), 5.23 (s, 1H), 4.55-4.53 (m, 1H), 3.49 (s, 1H), 2.74-2.64 (m, 1H), 2.40-2.38 (m, 1H). F-NMR: (400 MHz, DMSO-d₆, ppm): δ −51.987 (s, 1F).

### Example 9

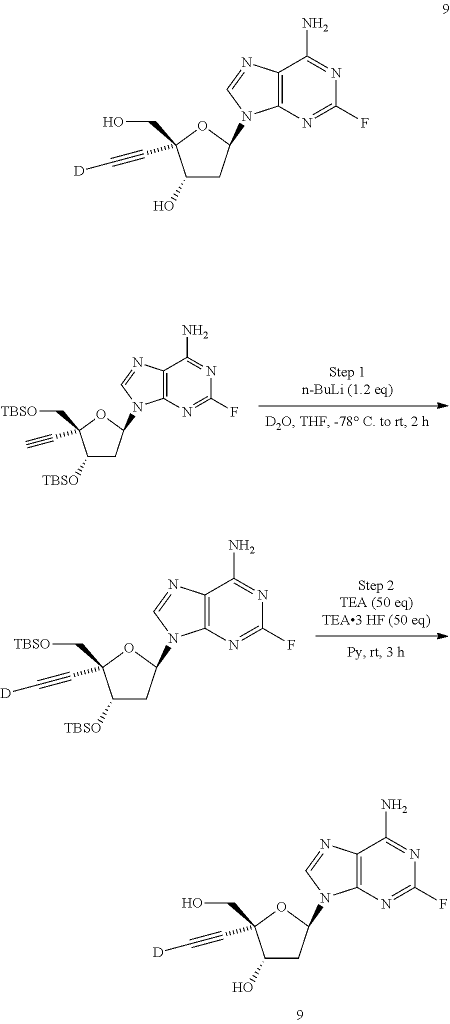 [SMILES: [2H]C#C[C@]1(CO)O[C@@H](n2cnc3c(N)nc(F)nc32)C[C@@H]1O ; C#C[C@]1(CO[Si](C)(C)C(C)(C)C)O[C@@H](n2cnc3c(N)nc(F)nc32)C[C@@H]1O[Si](C)(C)C(C)(C)C ; [2H]C#C[C@]1(CO[Si](C)(C)C(C)(C)C)O[C@@H](n2cnc3c(N)nc(F)nc32)C[C@@H]1O[Si](C)(C)C(C)(C)C ; [2H]C#C[C@]1(CO)O[C@@H](n2cnc3c(N)nc(F)nc32)C[C@@H]1O]

### Synthesis of (2R,3S,5R)-5-(6-amino-2-fluoro-9H-purin-9-yl)-2-(²H)ethynyl-2-(hydroxymethyl)oxolan-3-ol (9)

9-((2R,4S,5R)-4-((tert-butyldimethylsilyl)oxy)-5-(((tert-butyldimethylsilyl)oxy)methyl)-5-ethynyltetrahydrofuran-2-yl)-2-fluoro-9H-purin-6-amine was prepared according to the protocol outlined in EXAMPLE 7.

Step 1: To a mixture of 9-((2R,4S,5R)-4-((tert-butyldimethylsilyl)oxy)-5-(((tert-butyldimethylsilyl)oxy)methyl)-5-ethynyltetrahydrofuran-2-yl)-2-fluoro-9H-purin-6-amine (65 mg, 0.125 mmol) in THF (2 mL) was added n-BuLi (0.050 mL, 0.125 mmol) under a argon atmosphere at −78° C. The resulting mixture was stirred for 1 h at −78° C. Then deuterium oxide (0.2 mL, 10.79 mmol) was added to the mixture, the resulting mixture was warmed to 25° C. slowly and stirred for 1.5 h. The major was desired product detected on LCMS. The reaction mixture was quenched with NH₄Cl (20 mL), extracted with EtOAc (100 mL), the organic layer was washed with brine (sat., 20 mL) and dried with Na₂SO₄, evaporated under reduced pressure. The residue was purified with Prep-TLC, eluted with EtOAc/petroleum ether (1/1) to afford 9-[(2R,4S,5R)-4-[(tert-butyldimethylsilyl)oxy]-5-{[(tert-butyldimethylsilyl)oxy]methyl}-5-(²H)ethynyloxolan-2-yl]-2-fluoro-9H-purin-6-amine (45 mg, 0.086 mmol, 69.1% yield) as an off-white solid. MS ESI calculated for C₂₄H₄₀DFN₅O₃Si₂[M+H]⁺ 523.79, found 523.50. ¹H-NMR: (300 MHz, d₆-DMSO, ppm) δ 8.20 (s, 1H), 7.78 (s, 2H), 6.21 (dd, J=7.7, 4.1 Hz, 1H), 4.89 (t, J=7.0 Hz, 1H), 3.77 (d, J=11.1 Hz, 1H), 3.60 (d, J=11.2 Hz, 1H), 2.98-2.88 (m, 1H), 2.45-2.32 (m, 1H), 0.88 (s, 9H), 0.72 (s, 9H), −0.07 (s, 3H), −0.19 (s, 3H).

Step 2: To a mixture of 9-[(2R,4S,5R)-4-[(tert-butyldimethylsilyl)oxy]-5-{[(tert-butyldimethylsilyl)oxy]methyl}-5-(²H)ethynyloxolan-2-yl]-2-fluoro-9H-purin-6-amine (45 mg, 0.086 mmol) in Py (2 mL) was added TEA (1.200 mL, 8.61 mmol) under an argon atmosphere at 20° C., the resulting mixture was stirred for 5 min at 20° C. Then triethylamine trihydrofluoride (1388 mg, 8.61 mmol) was added at 0° C. The resulting mixture was stirred for 3 h at 25° C. The major was desired product on LCMS/TLC. The reaction mixture was concentrated under reduced pressure. The residue was purified with Prep-TLC, eluted with DCM/MeOH (10/1) to afford (2R,3S,5R)-5-(6-amino-2-fluoro-9H-purin-9-yl)-2-(²H)ethynyl-2-(hydroxymethyl)oxolan-3-ol, 9, (20 mg, 0.065 mmol, 75% yield) as an off-white solid. MS ESI calculated for C₁₂H₁₂DFN₅O3 [M+H]⁺ 295.10, found 295.10. ¹H-NMR: (400 MHz, d₆-DMSO, ppm) δ 8.31 (s, 1H), 7.84 (s, 2H), 6.25 (dd, J=7.2, 5.0 Hz, 1H), 5.56 (d, J=5.4 Hz, 1H), 5.29 (dd, J=6.7, 5.6 Hz, 1H), 4.58-4.56 (m, 1H), 3.66 (dd, J=11.9, 5.7 Hz, 1H), 3.56 (dd, J=11.9, 6.7 Hz, 1H), 3.51 (s, 0.4H), 2.71-2.69 (m, 1H), 2.43-2.41 (m, 1H). F-NMR: (376 MHz, d₆-DMSO, ppm) δ −51.980 (s, 1F).

### Example 10

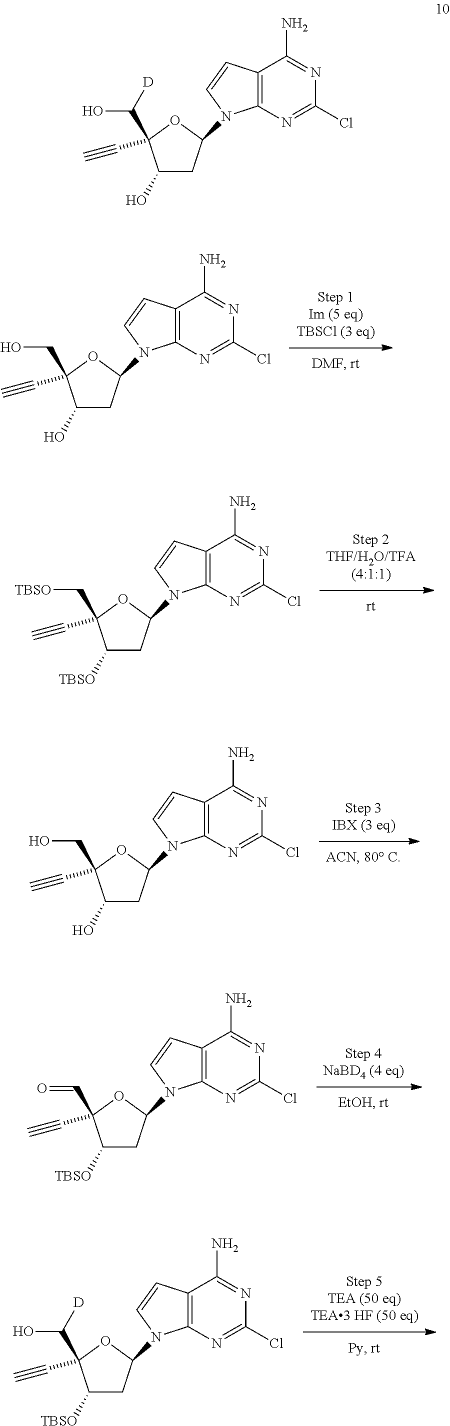 [SMILES: [2H]C(O)[C@@]1(C#C)O[C@@H](n2ccc3c(N)nc(Cl)nc32)C[C@@H]1O ; C#C[C@]1(CO)O[C@@H](n2ccc3c(N)nc(Cl)nc32)C[C@@H]1O ; C#C[C@]1(CO[Si](C)(C)C(C)(C)C)O[C@@H](n2ccc3c(N)nc(Cl)nc32)C[C@@H]1O[Si](C)(C)C(C)(C)C ; C#C[C@]1(CO)O[C@@H](n2ccc3c(N)nc(Cl)nc32)C[C@@H]1O ; C#C[C@]1(C=O)O[C@@H](n2ccc3c(N)nc(Cl)nc32)C[C@@H]1O[Si](C)(C)C(C)(C)C ; [2H]C(O)[C@@]1(C#C)O[C@@H](n2ccc3c(N)nc(Cl)nc32)C[C@@H]1O[Si](C)(C)C(C)(C)C]

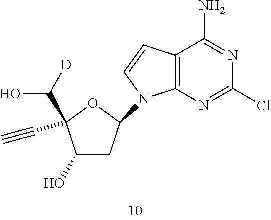 [SMILES: [2H]C(O)[C@@]1(C#C)O[C@@H](n2ccc3c(N)nc(Cl)nc32)C[C@@H]1O]

### Synthesis of (2R,3S,5R)-5-{4-amino-2-chloro-7H-pyrrolo[2,3-D]pyrimidin-7-yl}-2-ethynyl-2-[hydroxy(²H)methyl]oxolan-3-ol (10)

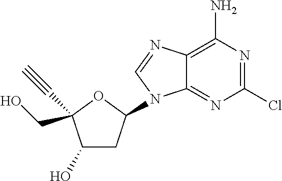 [SMILES: C#C[C@]1(CO)O[C@@H](n2cnc3c(N)nc(Cl)nc32)C[C@@H]1O]

(2R,3S,5R)-5-(4-amino-2-chloro-7H-pyrrolo[2,3-d]pyrimidin-7-yl)-2-ethynyl-2-(hydroxymethyl)tetrahydrofuran-3-ol was prepared according to the protocols described in PCT publication WO2015/148746.

Step 1:

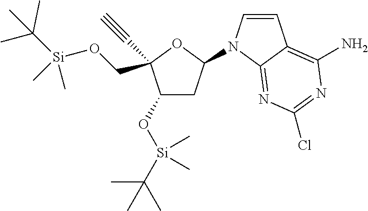 [SMILES: C#C[C@]1(CO[Si](C)(C)C(C)(C)C)O[C@@H](n2ccc3c(N)nc(Cl)nc32)C[C@@H]1O[Si](C)(C)C(C)(C)C]

To a solution of imidazole (441 mg, 6.48 mmol) and (2R,3S,5R)-5-(4-amino-2-chloro-7H-pyrrolo[2,3-d]pyrimidin-7-yl)-2-ethynyl-2-(hydroxymethyl)tetrahydrofuran-3-ol (400 mg, 1.296 mmol) in DMF (0.4 mL) was added TBSCl (586 mg, 3.89 mmol) at 0° C. under the argon atmosphere. The resulting mixture was stirred for 5 h at 25° C. The reaction progress was monitored by LCMS. The major was desired product, the mixture was concentrated under the vacuum. The crude was purified by silica gel column chromatography, eluted with PE/EA (4/1) to give 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (670 mg, 1.218 mmol, 94% yield) as a white solid. MS ESI calculated for C₂₅H₄₂ClN₄O₃Si₂ [M+H]⁺ 537.24, found 537.40. ¹H-NMR: (400 MHz, d₆-DMSO, ppm) δ 7.51 (s, 2H), 7.25 (d, J=3.7 Hz, 1H), 6.58 (d, J=3.7 Hz, 1H), 6.32 (dd, J=7.3, 5.4 Hz, 1H), 4.72 (t, J=6.5 Hz, 1H), 3.76 (d, J=11.0 Hz, 1H), 3.62 (d, J=11.0 Hz, 1H), 3.49 (s, 1H), 2.73 (dt, J=12.5, 6.0 Hz, 1H), 2.35 (dt, J=13.6, 7.1 Hz, 1H), 0.90 (s, 9H), 0.82 (s, 9H), 0.12 (d, J=3.3 Hz, 6H), −0.08-0.01 (m, 6H).

Step 2: A mixture of TFA (1 mL, 12.98 mmol), THF (4 mL) and water (1 mL) were formed a mixture solvent and cooled to 0° C. 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine (100 mg, 0.186 mmol) in a 25 mL flask was cooled to 0° C., to this was added the mixture solvent at 0° C. The resulting mixture was stirred for 1.5 h at 20° C. The major was desired product, trace starting material was left. The mixture was concentrated under reduced pressure and purified with Prep-TLC, eluted with DCM/MeOH (12/1) to afford ((2R,3S,5R)-5-(4-amino-2-chloro-7H-pyrrolo[2,3-d]pyrimidin-7-yl)-3-((tert-butyldimethylsilyl)oxy)-2-ethynyltetrahydrofuran-2-yl)methanol (55 mg, 0.124 mmol, 66.4% yield) as an off-white solid. MS ESI calculated for C₁₉H₂₈ClN₄O₃Si [M+H]⁺ 423.15, found 423.20. ¹H-NMR: (300 MHz, d₆-DMSO, ppm) δ 7.52 (s, 2H), 7.31 (d, J=3.7 Hz, 1H), 6.58 (d, J=3.7 Hz, 1H), 6.35 (t, J=6.6 Hz, 1H), 5.28 (t, J=6.1 Hz, 1H), 4.64 (t, J=5.9 Hz, 1H), 3.64-3.40 (m, 3H), 2.59-2.56 (m, 1H), 2.37-2.23 (m, 1H), 0.88 (s, 9H), 0.10 (d, J=3.3 Hz, 6H).

Step 3: To a mixture of ((2R,3S,5R)-5-(4-amino-2-chloro-7H-pyrrolo[2,3-d]pyrimidin-7-yl)-3-((tert-butyldimethylsilyl)oxy)-2-ethynyltetrahydrofuran-2-yl)methanol (55 mg, 0.130 mmol) in ACN (4 mL) was added IBX (109 mg, 0.390 mmol) under a argon atmosphere at 20° C. The resulting mixture was stirred for 30 min at 80° C. The major was desired product. The reaction mixture was cooled to 20° C. and filtered, the filtrate was concentrated under reduced pressure to afford (2S,3S,5R)-5-(4-amino-2-chloro-7H-pyrrolo[2,3-d]pyrimidin-7-yl)-3-((tert-butyldimethylsilyl)oxy)-2-ethynyltetrahydrofuran-2-carbaldehyde (60 mg, 0.114 mmol, 88% yield) as an off-white solid crude used to next step directly without purification. MS ESI calculated for C₁₉H₂₆ClN₄O₃Si [M+H]⁺ 421.14, found 421.25.

Step 4: To a mixture of (2S,3S,5R)-5-(4-amino-2-chloro-7H-pyrrolo[2,3-d]pyrimidin-7-yl)-3-((tert-butyldimethylsilyl)oxy)-2-ethynyltetrahydrofuran-2-carbaldehyde (60 mg, 0.114 mmol) in EtOH (1 mL) was added sodium borodeuteride (19.09 mg, 0.456 mmol) under a argon atmosphere at 0° C. The resulting mixture was stirred for 60 min at 25° C. The major was desired product on LCMS. The reaction mixture was cooled to 0° C., quenched with HOAc (1 d), concentrated under reduced pressure. The residue was purified with Prep-TLC, eluted with DCM/MeOH (15/1) to afford [(2R,3S,5R)-5-{4-amino-2-chloro-7H-pyrrolo[2,3-d]pyrimidin-7-yl}-3-[(tert-butyldimethylsilyl)oxy]-2-ethynyloxolan-2-yl](²H₁)methanol (21 mg, 0.047 mmol, 41.3% yield) as an off-white solid. MS ESI calculated for C₁₉H₂₇DClN₄O₃Si [M+H]⁺ 424.16, found 424.10. ¹H-NMR: (300 MHz, d₆-DMSO, ppm) δ 7.53 (s, 2H), 7.33 (d, J=3.7 Hz, 1H), 6.59 (d, J=3.6 Hz, 1H), 6.37 (t, J=6.6 Hz, 1H), 5.28 (d, J=6.1 Hz, 1H), 4.65 (t, J=5.9 Hz, 1H), 3.57 (d, J=5.6 Hz, 1H), 3.47 (d, J=4.7 Hz, 1H), 2.60-2.58 (m, 1H), 2.38-2.27 (m, 1H), 0.90 (s, 9H), 0.11 (d, J=3.3 Hz, 6H).

Step 5: To a mixture of [(2R,3S,5R)-5-{4-amino-2-chloro-7H-pyrrolo[2,3-d]pyrimidin-7-yl}-3-[(tert-butyldimethylsilyl)oxy]-2-ethynyloxolan-2-yl](²H₁)methanol (20 mg, 0.047 mmol) in Py (0.5 mL) was added Et₃N (0.329 mL, 2.359 mmol) under a argon atmosphere at 20° C., the resulting mixture was stirred for 5 min at 20° C. Then triethylaminetrihydrofluoride (380 mg, 2.359 mmol) was added at 0° C. The resulting mixture was stirred for 15 h at 25° C. The major was desired product on LCMS/TLC. The reaction mixture was concentrated under reduced pressure. The residue was purified with Prep-TLC, eluted with DCM/MeOH (10/1) to afford a crude product. The crude was purified with Prep-HPLC with the following conditions: Column: Atlantis Prep T3 OBD Column, 19*250 mm, 10 u; Mobile Phase A: Water (10 mM NH₄HCO₃), Mobile Phase B: ACN; Flow rate: 20 mL/min; Gradient: 16% B to 45% B in 9 min; 210 nm; Rt: 7.12 min to afford (2R,3S,5R)-5-{4-amino-2-chloro-7H-pyrrolo[2,3-d]pyrimidin-7-yl}-2-ethynyl-2-[hydroxy(²H)methyl]oxolan-3-ol, 10, (8 mg, 0.025 mmol, 52.0% yield) as an off-white solid. MS ESI calculated for C₁₃H₁₃DClN₄O₃ [M+H]⁺ 310.07, found 310.10. ¹H-NMR: (400 MHz, d₆-DMSO, ppm) δ 7.54 (s, 2H), 7.34 (d, J=3.7 Hz, 1H), 6.61 (d, J=3.7 Hz, 1H), 6.42 (t, J=6.4 Hz, 1H), 5.52 (d, J=5.4 Hz, 1H), 5.24 (d, J=6.0 Hz, 1H), 4.48 (q, J=6.4 Hz, 1H), 3.65-3.45 (m, 2H), 2.56-2.44 (m, 1H), 2.36-2.33 (m, 1H).

### Example 11

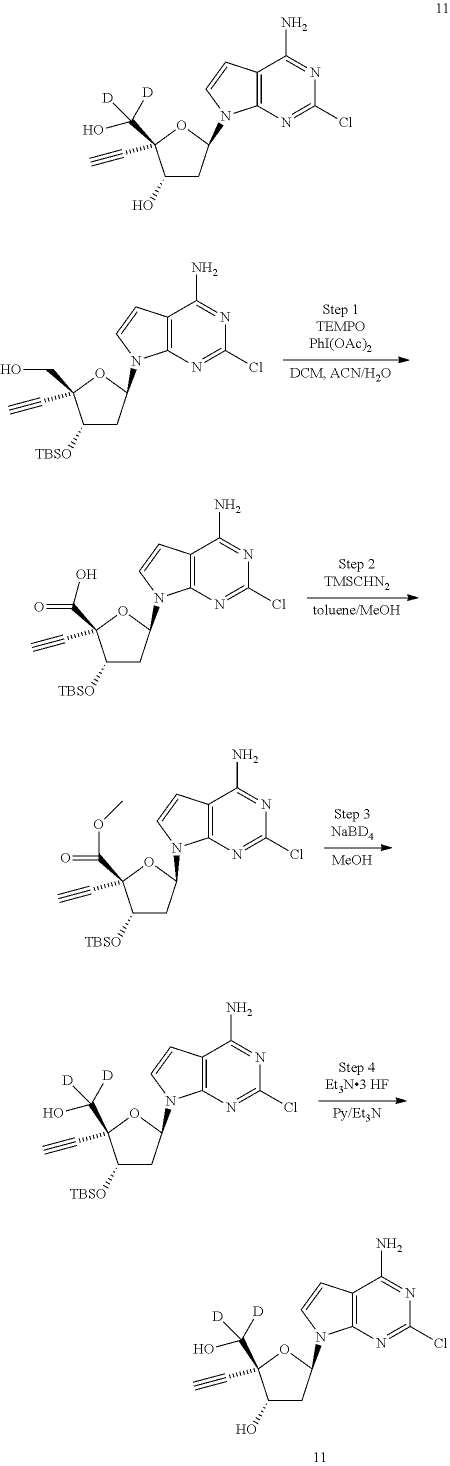

### Synthesis of (2R,3S,5R)-5-{4-amino-2-chloro-7H-pyrrolo[2,3-d]pyrimidin-7-yl}-2-ethynyl-2-[hydroxy(²H₂)methyl]oxolan-3-ol (11)

((2R,3S,5R)-5-(4-amino-2-chloro-7H-pyrrolo[2,3-d]pyrimidin-7-yl)-3-((tert-butyldimethylsilyl)oxy)-2-ethynyltetrahydrofuran-2-yl)methanol was prepared according to the protocol described in EXAMPLE 10, steps 1 and 2.

Step 1: To a solution of ((2R,3S,5R)-5-(4-amino-2-chloro-7H-pyrrolo[2,3-d]pyrimidin-7-yl)-3-((butyldimethylsilyl)oxy)-2-ethynyltetrahydrofuran-2-yl)methanol (140 mg, 0.331 mmol) in DCM (3.5 mL) were added TEMPO (10.34 mg, 0.066 mmol) and BAIB (235 mg, 0.728 mmol) at 20° C. After the mixture was stirred for 2 h, a mixture of MeCN-water (0.05 mL/0.05 mL) was added to the reaction mixture, and the mixture was stirred for 4 h. The reaction progress was monitored by LCMS. The mixture was gradually poured into 0.5 N KOH (7 mL). After stirring for 10 minutes at 20° C., the mixture was extracted with CH₂Cl₂ (3×7 mL). Combined organic layers were washed with water (7 mL), combined water layers were acidified carefully under cooling to pH=2 with 2 N HCl. The lade-down sediment was filtered, washed with water and dried to give (2S,3S,5R)-5-(4-amino-2-chloro-7H-pyrrolo[2,3-d]pyrimidin-7-yl)-3-((tert-butyldimethyl silyl)oxy)-2-ethynyltetrahydrofuran-2-carboxylic acid (130 mg, 0.238 mmol, 71.9% yield) as a brown solid. MS ESI calculated for C₁₉H₂₆ClN₄O₄Si [M+H]⁺ 437.73, found 437.30. ¹H-NMR: (400 MHz, DMSO-d₆, ppm) δ 13.60 (s, 1H), 7.57 (s, 2H), 7.36 (d, J=3.7 Hz, 1H), 6.64 (d, J=3.7 Hz, 1H), 6.52 (t, J=7.1 Hz, 1H), 4.85 (dd, J=5.1, 3.5 Hz, 1H), 3.66 (s, 1H), 2.59-2.57 (m, 1H), 2.40-2.30 (m, 1H), 0.94 (s, 9H), 0.17 (d, J=1.0 Hz, 6H).

Step 2: To a solution of (2S,3S,5R)-5-(4-amino-2-chloro-7H-pyrrolo[2,3-d]pyrimidin-7-yl)-3-((tert-butyldimethylsilyl)oxy)-2-ethynyltetrahydrofuran-2-carboxylic acid (80 mg, 0.183 mmol) in toluene (2.5 mL)-MeOH (2 mL) was added (diazomethyl)trimethylsilane (2.105 mL, 4.21 mmol) at 20° C., and the mixture was stirred for 5 h at 20° C. The reaction progress was monitored by LCMS. After the reaction complete, the mixture was concentrated under the vacuum. The crude was purified by column chromatography on silica gel [100-200], eluted with EtOAc/petroleum ether (1/1) to give (2S,3S,5R)-methyl 5-(4-amino-2-chloro-7H-pyrrolo[2,3-d]pyrimidin-7-yl)-3-((tert-butyldimethylsilyl)oxy)-2-ethynyltetrahydrofuran-2-carboxylate (30 mg, 0.035 mmol, 36.7% yield) as an off-white solid. MS ESI calculated for C₂₀H₂₈ClN₄O₄S [M+H]⁺ 451.15, found 451.10. ¹H-NMR: (300 MHz, CDCl₃, ppm): δ 7.36 (d, J=3.7 Hz, 1H), 6.78 (t, J=6.7 Hz, 1H), 6.41 (d, J=3.7 Hz, 1H), 5.50 (s, 2H), 4.85 (dd, J=5.5, 4.0 Hz, 1H), 3.86 (s, 3H), 2.71-2.55 (m, 2H), 2.51-2.49 (m, 1H), 0.96 (s, 9H), 0.17-0.16 (m, 6H).

Step 3: To a stirred solution of (2S,3S,5R)-methyl 5-(4-amino-2-chloro-7H-pyrrolo[2,3-d]pyrimidin-7-yl)-3-((tert-butyldimethylsilyl)oxy)-2-ethynyltetrahydrofuran-2-carboxylate (45 mg, 0.100 mmol) in MeOH (3.5 mL) was added sodium borodeuteride (16.71 mg, 0.399 mmol) batchwise at 0° C. The resulting solution was stirred at 20° C. for 2 h, adjusted pH value to 7 with acetic acid and extracted with CHCl₃, the organic layer was washed with sat. NaCl, then concentrated under reduced pressure. The residue was purified by silica-gel column chromatography, eluting with 5% MeOH in DCM to afford [(2R,3S,5R)-5-{4-amino-2-chloro-7H-pyrrolo[2,3-d]pyrimidin-7-yl}-3-[(tert-butyldimethylsilyl)oxy]-2-ethynyloxolan-2-yl](²H₂)methanol (30 mg, 0.067 mmol, 67.2% yield) as an off-white solid. MS ESI calculated for C₁₉H₂₆D₂ClN₄O₃Si [M+H]⁺ 425.17, found 425.20. ¹H-NMR: (400 MHz, CDCl₃, ppm): δ 7.03 (d, J=3.6 Hz, 1H), 6.37 (d, J=3.7 Hz, 1H), 6.29 (dd, J=8.5, 6.0 Hz, 1H), 5.69 (s, 2H), 4.79 (dd, J=5.8, 2.7 Hz, 1H), 3.19-3.02 (m, 1H), 2.61 (s, 1H), 2.36-2.26 (m, 1H), 0.97 (s, 9H), 0.18 (d, J=9.4 Hz, 6H).

Step 4: Into a solution of [(2R,3S,5R)-5-{4-amino-2-chloro-7H-pyrrolo[2,3-d]pyrimidin-7-yl}-3-[(tert-butyldimethylsilyl)oxy]-2-ethynyloxolan-2-yl](²H₂)methanol (10 mg, 0.024 mmol) in pyridine (0.5 mL) was added triethylamine (119 mg, 1.176 mmol) under an argon atmosphere at 20° C. Then triethylamine trihydrofluoride (190 mg, 1.176 mmol) was added at 0° C. The mixture was stirred at 20° C. for 6 h. After the reaction completed, the mixture was concentrated under the vacuum. The residue was purified by Pre-HPLC Column: XBridge Shield RP18 OBD Column, 5 um, 19*150 mm; Mobile Phase A: Water (10 mM NH₄HCO₃), Mobile Phase B: ACN; Flow rate: 20 mL/min; Gradient: 5% B to 80% B in 10 min; 254 nm; Rt: 9.75 min to give (2R,3S,5R)-5-{4-amino-2-chloro-7H-pyrrolo[2,3-d]pyrimidin-7-yl}-2-ethynyl-2-[hydroxy(²H₂)methyl]oxolan-3-ol, 10, (6 mg, 0.018 mmol, 78% yield) as an off-white solid. MS ESI calculated for C₁₃H₁₂D₂ClN₄O₃ [M+H]⁺ 311.08, found 311.1. ¹H-NMR: (400 MHz, DMSO-d₆, ppm): δ 7.52 (s, 2H), 7.31 (d, J=3.7 Hz, 1H), 6.58 (d, J=3.6 Hz, 1H), 6.39 (t, J=6.4 Hz, 1H), 5.49 (d, J=5.1 Hz, 1H), 5.20 (d, J=4.5 Hz, 1H), 4.46 (q, J=6.2 Hz, 1H), 3.45 (s, 1H), 2.53-2.42 (m, 1H), 2.33-2.31 (m, 1H).

### Example 12

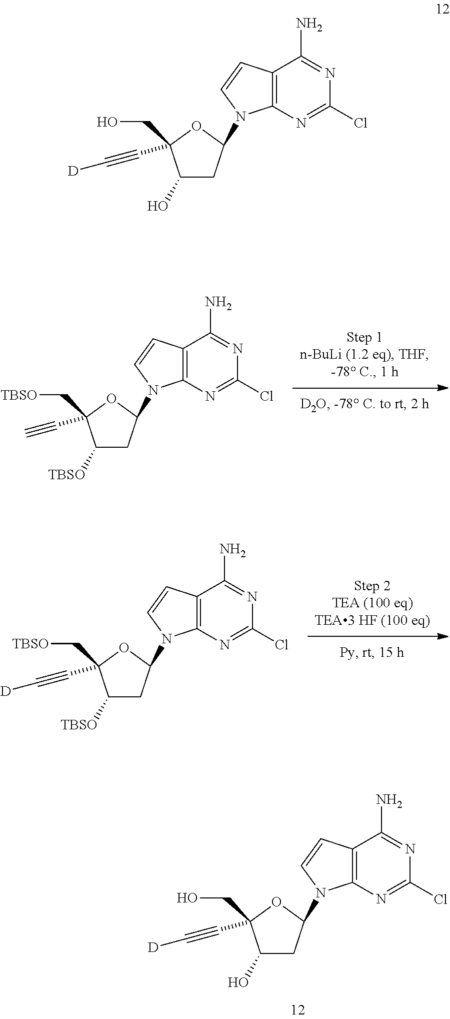 [SMILES: [2H]C#C[C@]1(CO)O[C@@H](n2ccc3c(N)nc(Cl)nc32)C[C@@H]1O ; C#C[C@]1(CO[Si](C)(C)C(C)(C)C)O[C@@H](n2ccc3c(N)nc(Cl)nc32)C[C@@H]1O[Si](C)(C)C(C)(C)C ; [2H]C#C[C@]1(CO[Si](C)(C)C(C)(C)C)O[C@@H](n2ccc3c(N)nc(Cl)nc32)C[C@@H]1O[Si](C)(C)C(C)(C)C ; [2H]C#C[C@]1(CO)O[C@@H](n2ccc3c(N)nc(Cl)nc32)C[C@@H]1O]

### Synthesis of (2R,3S,5R)-5-{4-amino-2-chloro-7H-pyrrolo[2,3-d]pyrimidin-7-yl}-2-(²H)ethynyl-2-(hydroxymethyl)oxolan-3-ol (12)

7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine was prepared according to the protocol described in EXAMPLE 10, step 1.

Step 1: To a mixture of 7-((2R,4S,5R)-4-((tert-butyldimethylsilyl)oxy)-5-(((tert-butyldimethylsilyl)oxy)methyl)-5-ethynyltetrahydrofuran-2-yl)-2-chloro-7H-pyrrolo[2,3-d]pyrimidin-4-amine 45 mg, 0.084 mmol) in THF (2 mL) was added n-BuLi (0.034 mL, 0.084 mmol) under a argon atmosphere at −78° C. The resulting mixture was stirred for 1 h at −78° C. Then deuterium oxide (0.1 mL, 10.79 mmol) was added to the mixture, the resulting mixture was warmed to 25° C. slowly and stirred for 1.5 h. The major was desired product detected on LCMS. The reaction mixture was quenched with NH₄Cl (20 mL), extracted with EA (100 mL), the organic layer was washed with brine (sat., 20 mL) and dried with Na₂SO₄, evaporated under reduced pressure. The residue was purified with Prep-TLC, eluted with EA/PE (1/1) to afford 7-[(2R,4S,5R)-4-[(tert-butyldimethyl silyl)oxy]-5-{[(tert-butyldimethylsilyl)oxy]methyl}-5-(²H)ethynyloxolan-2-yl]-2-chloro-7H-pyrrolo[2,3-d]pyrimidin-4-amine (35 mg, 0.064 mmol, 76% yield) as an off-white solid. MS ESI calculated for C₂₅H₄₁DClN₄O₃Si₂ [M+H]⁺ 538.25, found 538.20. ¹H-NMR: (400 MHz, CDCl₃, ppm) δ 7.25 (d, J=3.7 Hz, 1H), 6.61 (dd, J=7.3, 4.5 Hz, 1H), 6.38 (d, J=3.7 Hz, 1H), 5.57 (s, 2H), 5.30 (s, 1H), 4.77 (t, J=7.1 Hz, 1H), 3.93 (d, J=11.2 Hz, 1H), 3.79 (d, J=11.2 Hz, 1H), 2.64-2.45 (m, 2H), 1.28-1.23 (m, 1H), 0.92 (d, J=7.1 Hz, 18H), 0.16-0.04 (m, 12H).

Step 2: To a mixture of 7-[(2R,4S,5R)-4-[(tert-butyldimethylsilyl)oxy]-5-{[(tert-butyldimethylsilyl)oxy]methyl}-5-(²H)ethynyloxolan-2-yl]-2-chloro-7H-pyrrolo[2,3-d]pyrimidin-4-amine (30 mg, 0.056 mmol) in pyridine (1 mL) was added TEA (0.777 mL, 5.57 mmol) under a argon atmosphere at 20° C., the resulting mixture was stirred for 5 min at 20° C. Then triethylamine trihydrofluoride (899 mg, 5.57 mmol) was added at 0° C. The resulting mixture was stirred for 15 h at 25° C. The major was desired product on LCMS/TLC. The reaction mixture was concentrated under reduced pressure. The residue was purified with Prep-TLC, eluted with DCM/MeOH (10/1) to afford a crude product. The crude was crystallized from CDCl₃/hexane (1/1) to afford (2R,3S,5R)-5-{4-amino-2-chloro-7H-pyrrolo[2,3-d]pyrimidin-7-yl}-2-(²H)ethynyl-2-(hydroxymethyl)oxolan-3-ol, 12, (15 mg, 0.046 mmol, 83% yield) as an off-white solid. MS ESI calculated for C₁₃H₁₃DClN₄O₃ [M+H]⁺ 310.07, found 309.85. ¹H-NMR: (400 MHz, d₆-DMSO, ppm) δ 7.54 (s, 2H), 7.34 (d, J=3.7 Hz, 1H), 6.61 (d, J=3.7 Hz, 1H), 6.41 (t, J=6.5 Hz, 1H), 5.51 (d, J=5.5 Hz, 1H), 5.26 (t, J=6.1 Hz, 1H), 4.48 (q, J=6.3 Hz, 1H), 3.67-3.50 (m, 2H), 3.48 (s, 0.4H), 2.60-2.44 (m, 1H), 2.36-2.33 (m, 1H).

### Example 13

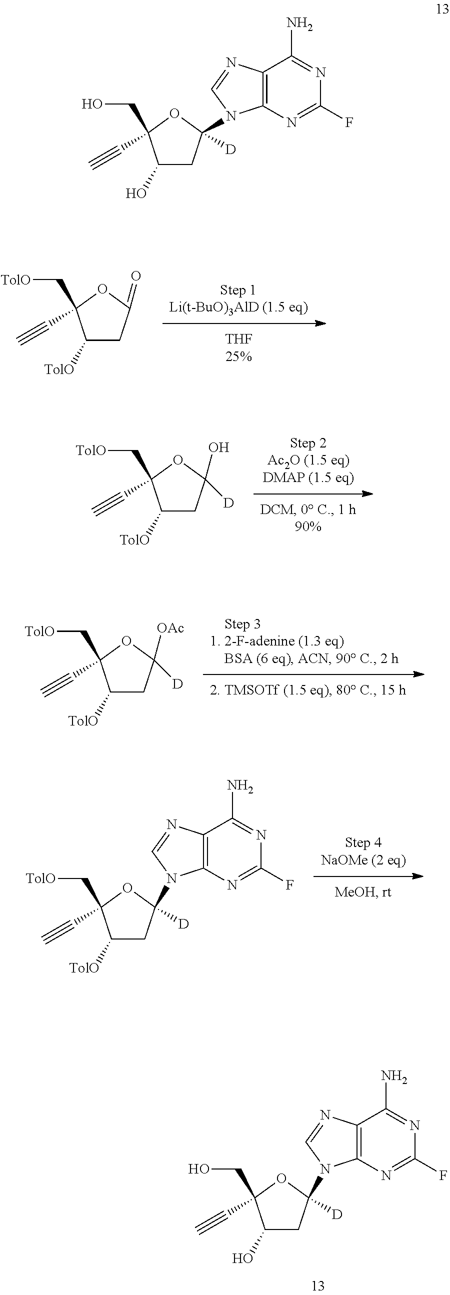

### Synthesis of (2R,3S,5R)-5-(6-amino-2-fluoro-9H-purin-9-yl)-2-ethynyl-2-(hydroxymethyl)(5-²H)oxolan-3-ol (13)

(2R,3S)-2-ethynyl-2-(((4-methylbenzoyl)oxy)methyl)-5-oxotetrahydrofuran-3-yl 4-methylbenzoate was prepared according to the protocol described in patent application U.S. Pat. No. 7,339,053.

Step 1: To a mixture of (2R,3S)-2-ethynyl-2-(((4-methylbenzoyl)oxy)methyl)-5-oxotetrahydrofuran-3-yl 4-methylbenzoate (500 mg, 1.274 mmol) in THF (10 mL) was added lithium tri-tert-butoxyaluminodeuteride (488 mg, 1.911 mmol) under an argon atmosphere at −20° C. The resulting mixture was warmed to 20° C. and stirred for 3 h, 32% desired product was detected by LCMS. The reaction mixture was quenched with HOAc, extracted with EA (2×100 mL), the combined organic layer was washed with HCl (2 N, 50 mL), water (2×50 mL) and brine (sat., 50 mL), dried over anhydrous Na₂SO₄, and then evaporated under reduced pressure. The residue was purified with silica gel column chromatography, eluted with 25% EA in PE to afford [(2R,3S)-2-ethynyl-5-hydroxy-3-[(4-methylphenyl)carbonyloxy](5-²H)oxolan-2-yl]methyl 4-methylbenzoate (130 mg, 0.296 mmol, 23.22% yield) as a colorless oil. MS ESI calculated for C₂₃H₂₁DNaO₆ [M+Na]⁺ 418.41, found 418.10. ¹H-NMR: (300 MHz, CDCl₃, ppm) δ 8.02-7.85 (m, 4H), 7.21 (m, 4H), 5.79 (t, J=7.1 Hz, 0.46H), 5.66 (dd, J=7.3, 3.3 Hz, 0.43H), 4.70-4.41 (m, 2H), 2.73-2.61 (m, 0.44H), 2.57 (d, J=9.2 Hz, 1H), 2.53-2.45 (m, 1H), 2.42-2.35 (m, 6H), 2.32 (dd, J=14.3, 3.3 Hz, 0.41H).

Step 2: Under an argon, to a mixed solution of [(2R,3S)-2-ethynyl-5-hydroxy-3-[(4-methylphenyl)carbonyloxy](5-²H)oxolan-2-yl]methyl 4-methylbenzoate (130 mg, 0.329 mmol) and DMAP (60.2 mg, 0.493 mmol) in anhydrous DCM (3 mL) was added Ac₂O (0.047 ml, 0.493 mmol) dropwise at 0° C. The reaction mixture was stirred for 1.0 h at 0° C. The reaction mixture was quenched by ice-cold water (3 mL). The resulting mixture was extracted with ice-cold DCM (2×30 mL). The combined organic layers were washed with saturated NaCl (30 mL), dried over anhydrous Na₂SO₄, and evaporated under reduced pressure. The residue was purified by silica gel column chromatography, eluted with 15% EA in PE to give the desired product [(2R,3S)-5-(acetyloxy)-2-ethynyl-3-[(4-methylphenyl)carbonyloxy](5-²H)oxolan-2-yl]methyl 4-methylbenzoate (130 mg, 0.282 mmol, 86% yield) as a colorless oil. MS ESI calculated for C₂₅H₂₃DNaO₇ [M+Na]⁺ 460.15, found 460.0. ¹H-NMR: (400 MHz, CDCl₃, ppm) δ 8.08-7.91 (m, 4H), 7.33-7.19 (m, 4H), 5.86 (t, J=7.5 Hz, 1H), 4.76 (d, J=11.7 Hz, 1H), 4.56 (d, J=11.7 Hz, 1H), 2.72-2.62 (m, 3H), 2.44 (d, J=12.2 Hz, 6H), 1.93 (s, 3H).

Step 3: To a mixture of 2-fluoro-9H-purin-6-amine (59.2 mg, 0.386 mmol) in ACN (4 mL) was added N,O-bis(trimethylsilyl)acetamide (363 mg, 1.783 mmol) at 20° C. under an argon atmosphere, the resulting mixture was stirred for 2 h at 90° C. Then cooled to 0° C., a solution of [(2R,3S)-5-(acetyloxy)-2-ethynyl-3-[(4-methylphenyl)carbonyloxy](5-²H)oxolan-2-yl]methyl 4-methylbenzoate (130 mg, 0.297 mmol) in ACN (1 mL) was added, then TMS-OTf (0.081 mL, 0.446 mmol) was added. The resulting mixture was stirred for 15 h at 80° C. No starting material was left, the major was desired product. The mixture was cooled to 0° C. and quenched with NaHCO₃ (sat., 20 mL), extracted with EA (100 mL), the organic layer was washed with brine (sat., 30 mL), dried with anhydrous Na₂SO₄ and evaporated under reduced pressure. The residue was purified with silica gel column chromatography, eluted with 30%-45% EA in PE to afford [(2R,3S,5S)-5-(6-amino-2-fluoro-9H-purin-9-yl)-2-ethynyl-3-[(4-methylphenyl)carbonyloxy](5-²H)oxolan-2-yl]methyl 4-methylbenzoate (40 mg, 0.072 mmol, 24.10% yield) (a isomer) as a off-white solid and [(2R,3S,5R)-5-(6-amino-2-fluoro-9H-purin-9-yl)-2-ethynyl-3-[(4-methylphenyl)carbonyloxy](5-²H)oxolan-2-yl]methyl 4-methylbenzoate (80 mg, 0.143 mmol, 48.2% yield) as a off-white solid. MS ESI calculated for C₂₈H₂₄DFN₅O₅ [M+H]⁺ 531.18, found 531.2. ¹H-NMR: (400 MHz, CDCl₃, ppm) δ 8.04 (d, J=8.3 Hz, 3H), 7.96-7.91 (m, 2H), 7.32-7.25 (m, 3H), 6.05 (dd, J=7.2, 5.3 Hz, 1H), 4.88 (d, J=12.1 Hz, 1H), 4.75-4.65 (m, 1H), 3.20 (dd, J=14.0, 7.3 Hz, 1H), 2.99-2.86 (m, 1H), 2.72 (s, 1H), 2.47 (s, 3H), 2.43 (s, 3H). ¹H-NMR-a isomer: (400 MHz, CDCl₃, ppm) δ 8.54 (s, 1H), 8.02-7.95 (m, 2H), 7.73-7.66 (m, 2H), 7.28 (dd, J=26.7, 8.0 Hz, 5H), 6.25 (s, 2H), 5.93 (dd, J=6.6, 2.8 Hz, 1H), 4.74 (d, J=11.9 Hz, 1H), 4.66 (d, J=11.8 Hz, 1H), 3.20 (dd, J=14.9, 6.6 Hz, 1H), 3.02 (dd, J=14.9, 2.8 Hz, 1H), 2.81 (s, 1H), 2.45 (d, J=18.0 Hz, 6H).

Step 4: To a mixture of [(2R,3S,5R)-5-(6-amino-2-fluoro-9H-purin-9-yl)-2-ethynyl-3-[(4-methylphenyl)carbonyloxy](5-²H)oxolan-2-yl]methyl 4-methylbenzoate (80 mg, 0.151 mmol) in MeOH (1 mL) was added sodium methoxide (0.302 ml, 0.302 mmol) under an argon atmosphere at 0° C. The resulting mixture was stirred for 30 min at 20° C. No starting material was left, the major was desired product. The reaction mixture was quenched with HOAc (2 drops), evaporated under reduced pressure. The residue was purified with Prep-TLC, eluted with DCM/MeOH (10/1) to afford a crude product. The crude was purified with Prep-HPLC with the following condition: Column: XBridge C18 OBD Prep Column 100 Å, 10 μm, 19 mm×250 mm; Mobile Phase A: Water (10 mM/L NH₄HCO₃), Mobile Phase B: ACN; Flow rate: 20 mL/min; Gradient: 60% B to 65% B in 9 min; 254/210 nm; Rt: 7.78 min to afford (2R,3S,5R)-5-(6-amino-2-fluoro-9H-purin-9-yl)-2-ethynyl-2-(hydroxymethyl)(5-²H)oxolan-3-ol (30 mg, 0.097 mmol, 64.2% yield) as an off-white solid. MS ESI calculated for C₁₂H₁₂DFN₅O₃ [M+H]⁺ 295.10, found 295.10. ¹H-NMR: (300 MHz, d₆-DMSO, ppm) δ 8.26 (s, 1H), 7.81 (s, 2H), 5.53 (d, J=5.4 Hz, 1H), 5.25 (t, J=6.2 Hz, 1H), 4.53 (q, J=6.5 Hz, 1H), 3.68-3.44 (m, 3H), 2.66 (dd, J=13.2, 6.8 Hz, 1H), 2.38 (dd, J=13.2, 7.0 Hz, 1H). F-NMR: (282 MHz, d₆-DMSO, ppm) 6-51.705 (S, 1F)

### Example 14

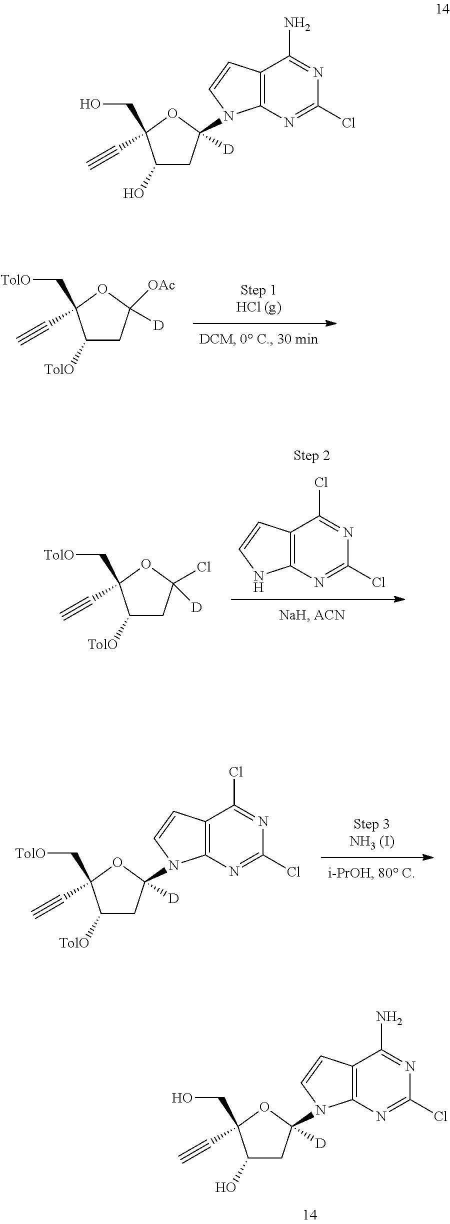

### Synthesis of (2R,3S,5R)-5-{4-amino-2-chloro-7H-pyrrolo[2,3-d]pyrimidin-7-yl}-2-ethynyl-2-(hydroxymethyl)(5-²H)oxolan-3-ol (14)

[(2R,3S)-5-(acetyloxy)-2-ethynyl-3-[(4-methylphenyl)carbonyloxy](5-²H)oxolan-2-yl]methyl 4-methylbenzoate was prepared according to the protocol described in EXAMPLE 13.

Step 1: [(2R,3S)-5-(acetyloxy)-2-ethynyl-3-[(4-methylphenyl)carbonyloxy](5-²H)oxolan-2-yl]methyl 4-methylbenzoate (200 mg, 0.457 mmol) was dried over P₂O₅ under vacuum overnight and then dissolved with dry DCM (5 mL) under an argon atmosphere at room temperature. The mixture was stirred until it became clear, then cooled to 0° C., slowly bubbled HCl gas while maintained the temperature at 0° C. The reaction progress was monitored by LCMS. After bubbling continued for 30 min, the started OAc-sugar was all consumed, then argon was bubbled into the mixture instead of HCl gas for 10 min to remove the dissolved residual HCl. The resulting DCM solution was partitioned between cold biphasic mixture of MTBE (50 mL) and aq NaHCO₃ (sat., 15 mL). The organic layer was collected, washed with cold aq NaHCO₃ (sat., 2×15 mL), dried over anhydrous MgSO₄ and filtered. The filtrate was concentrated under reduced pressure to give [(2R,3S)-5-chloro-2-ethynyl-3-[(4-methylphenyl)carbonyloxy](5-²H)oxolan-2-yl]methyl 4-methylbenzoate (200 mg, 0.435 mmol, 95% yield) as a light yellow oil, which was used to next step directly without further purification. MS ESI calculated for C₁ converted to OH C₂₃H₂₁DO₆ [M+Na]⁺ 418.15, found 418.05. ¹H-NMR: (400 MHz, CDCl₃, ppm) δ 8.13-7.91 (m, 4H), 7.34-7.18 (m, 4H), 6.02 (dd, J=8.9, 6.7 Hz, 0.32H), 5.86-5.80 (m, 0.46H), 4.83-4.52 (m, 2H), 3.09-2.77 (m, 2H), 2.70 (d, J=13.3 Hz, 1H), 2.48-2.39 (m, 6H).

Step 2: To a mixture of 2,4-dichloro-7h-pyrrolo[2,3-d]pyrimidine (109 mg, 0.580 mmol) in ACN (5 mL) was added NaH (23.19 mg, 0.580 mmol) under an argon atmosphere at 0° C. The resulting mixture was stirred for 30 min at 20° C. The solution was got to clear. Then a solution of [(2R,3S)-5-chloro-2-ethynyl-3-[(4-methylphenyl)carbonyloxy](5-²H)oxolan-2-yl]methyl 4-methylbenzoate (200 mg, 0.483 mmol) in ACN (2 mL) was added at 0° C. The resulting mixture was stirred for 2 h at 20° C. No starting material was left, the major was desired product. The mixture was quenched with HOAc (3 d), diluted with EA (200 mL), washed with water (50 mL) and brine (50 mL), the organic layer was dried with anhydrous Na₂SO₄, evaporated under reduced pressure. The residue was purified with 20% EA in PE to afford [(2R,3S,5R)-5-{2,4-dichloro-7H-pyrrolo[2,3-d]pyrimidin-7-yl}-2-ethynyl-3-[(4-methylphenyl)carbonyloxy](5-²H)oxolan-2-yl]methyl 4-methylbenzoate (120 mg, 0.212 mmol, 43.9% yield) as an off-white solid, and [(2R,3S,5S)-5-{2,4-dichloro-7H-pyrrolo[2,3-d]pyrimidin-7-yl}-2-ethynyl-3-[(4-methylphenyl)carbonyloxy](5-²H)oxolan-2-yl]methyl 4-methylbenzoate (80 mg, 0.141 mmol, 29.3% yield) (by-product) as an off-white solid. MS ESI calculated for C₂₉H₂₃DCl₂N₃O5 [M+H]⁺ 565.11, found 565.3. ¹H-NMR: (400 MHz, CDCl₃, ppm) δ 8.03 (d, J=7.9 Hz, 2H), 7.90 (d, J=7.9 Hz, 2H), 7.38-7.19 (m, 5H), 6.60-6.54 (m, 1H), 5.96 (t, J=6.8 Hz, 1H), 4.84 (d, J=12.0 Hz, 1H), 4.62 (d, J=11.8 Hz, 1H), 2.92 (qd, J=14.1, 6.4 Hz, 2H), 2.70 (s, 1H), 2.44 (d, J=9.8 Hz, 6H). ¹H-NMR-by-product: (400 MHz, CDCl₃, ppm) δ 8.01-7.94 (m, 2H), 7.93-7.81 (m, 3H), 7.30-7.24 (m, 4H), 6.69 (dd, J=3.9, 1.9 Hz, 1H), 5.86 (dq, J=6.2, 3.9, 3.0 Hz, 1H), 4.65 (qd, J=11.9, 1.9 Hz, 2H), 3.27-3.16 (m, 1H), 2.84-2.74 (m, 2H), 2.43 (d, J=3.1 Hz, 6H).

Step 3: A 100 mL-sealed tube was charged with a solution of [(2R,3S,5R)-5-{2,4-dichloro-7H-pyrrolo[2,3-d]pyrimidin-7-yl}-2-ethynyl-3-[(4-methylphenyl)carbonyloxy](5-²H)oxolan-2-yl]methyl 4-methylbenzoate (120 mg, 0.212 mmol) and ammonia (15 ml, 30.0 mmol) in 2-propanol (15 ml), the mixture was stirred for 15 h at 80° C. The major was desired product detected by LCMS. The mixture was concentrated under reduced pressure, and the residue was purified with silica gel column chromatography, eluted with DCM/MeOH (10/1) to afford a crude product. The crude was purified with Prep-HPLC with the following conditions: Column: XBridge C18 OBD Prep Column 100 Å, 10 μm, 19 mm×250 mm; Mobile Phase A: Water (10 MMOL/L NH₄HCO₃), Mobile Phase B: ACN; Flow rate: 20 mL/min; Gradient: 9% B to 30% B in 9 min; 254/210 nm; Rt: 7.87 min to afford (2R,3S,5R)-5-{4-amino-2-chloro-7H-pyrrolo[2,3-d]pyrimidin-7-yl}-2-ethynyl-2-(hydroxymethyl)(5-²H)oxolan-3-ol (20 mg, 0.063 mmol, 29.8% yield) as an off-white solid. MS ESI calculated for C₁₃H₁₃DClN₄O₃ [M+H]⁺ 310.07, found 310.20. ¹H-NMR: (300 MHz, d₆-DMSO, ppm) δ 7.51 (s, 2H), 7.31 (d, J=3.7 Hz, 1H), 6.58 (d, J=3.7 Hz, 1H), 5.48 (d, J=5.4 Hz, 1H), 5.22 (t, J=6.1 Hz, 1H), 4.46 (q, J=6.3 Hz, 1H), 3.65-3.41 (m, 3H), 2.45-2.42 (m, 1H), 2.32-2.28 (m, 1H).

TABLE 1

| Example No. | Structure |
|---|---|
| 1 | 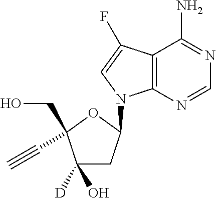 [SMILES: [2H][C@@]1(O)C[C@H](n2cc(F)c3c(N)ncnc32)O[C@]1(C#C)CO] |
| 2 | 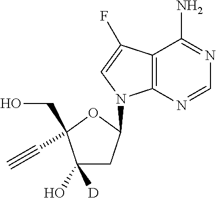 [SMILES: [2H][C@]1(O)C[C@H](n2cc(F)c3c(N)ncnc32)O[C@]1(C#C)CO] |
| 3 | 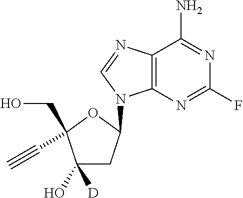 [SMILES: [2H][C@]1(O)C[C@H](n2cnc3c(N)nc(F)nc32)O[C@]1(C#C)CO] |
| 4 | 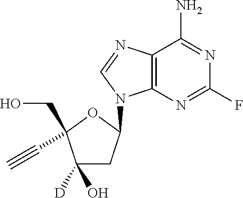 [SMILES: [2H][C@@]1(O)C[C@H](n2cnc3c(N)nc(F)nc32)O[C@]1(C#C)CO] |
| 5 | 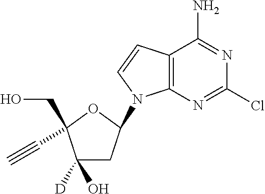 [SMILES: [2H][C@@]1(O)C[C@H](n2ccc3c(N)nc(Cl)nc32)O[C@]1(C#C)CO] |
| 6 | 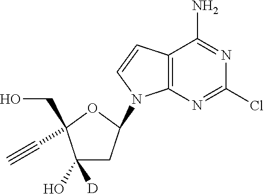 [SMILES: [2H][C@]1(O)C[C@H](n2ccc3c(N)nc(Cl)nc32)O[C@]1(C#C)CO] |
| 7 | 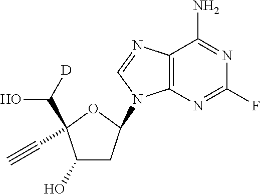 [SMILES: [2H]C(O)[C@@]1(C#C)O[C@@H](n2cnc3c(N)nc(F)nc32)C[C@@H]1O] |
| 8 | 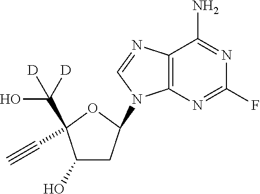 [SMILES: [2H]C([2H])(O)[C@@]1(C#C)O[C@@H](n2cnc3c(N)nc(F)nc32)C[C@@H]1O] |
| 9 | 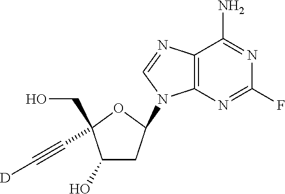 [SMILES: [2H]C#C[C@]1(CO)O[C@@H](n2cnc3c(N)nc(F)nc32)C[C@@H]1O] |
| 10 | 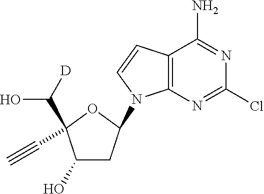 [SMILES: [2H]C(O)[C@@]1(C#C)O[C@@H](n2ccc3c(N)nc(Cl)nc32)C[C@@H]1O] |
| 11 | 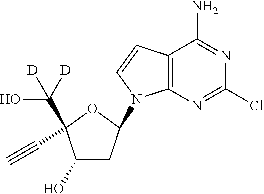 [SMILES: [2H]C([2H])(O)[C@@]1(C#C)O[C@@H](n2ccc3c(N)nc(Cl)nc32)C[C@@H]1O] |
| 12 | 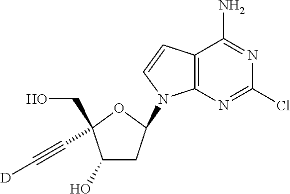 [SMILES: [2H]C#C[C@]1(CO)O[C@@H](n2ccc3c(N)nc(Cl)nc32)C[C@@H]1O] |
| 13 | 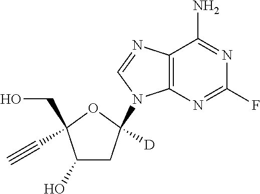 [SMILES: [2H][C@]1(n2cnc3c(N)nc(F)nc32)C[C@H](O)[C@@](C#C)(CO)O1] |
| 14 | 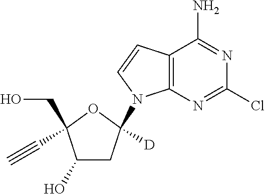 [SMILES: [2H][C@]1(n2ccc3c(N)nc(Cl)nc32)C[C@H](O)[C@@](C#C)(CO)O1] |

TABLE 2
The names of the corresponding salt-free form of the compounds exemplified above are,
respectively:

| | |
|---|---|
| 1 | (2R,3R,5R)-5-{4-amino-5-fluoro-7H-pyrrolo[2,3-d]pyrimidin-7-yl}-2-{[(tert- |
| | butyldiphenylsilyl)oxy]methyl}-2-ethynyl(3-²H)oxolan-3-ol |
| 2 | (2R,3S,5R)-5-{4-amino-5-fluoro-7H-pyrrolo[2,3-d]pyrimidin-7-yl}-2-ethynyl-2- |
| | (hydroxymethyl)(3-²H)oxolan-3-ol |
| 3 | (2R,3S,5R)-5-(6-amino-2-fluoro-9H-purin-9-yl)-2-ethynyl-2-(hydroxymethyl)(3- |
| | ²H)oxolan-3-ol |
| 4 | (2R,3R,5R)-5-(6-amino-2-fluoro-9H-purin-9-yl)-2-ethynyl-2-(hydroxymethyl)(3- |
| | ²H)oxolan-3-ol |
| 5 | (2R,3R,5R)-5-(4-amino-2-chloro-7H-pyrrolo[2,3-d]pyrimidin-7-yl)-2-ethynyl(3- |
| | ²H)-2-(hydroxymethyl)-tetrahydrofuran-3-ol |
| 6 | (2R,3S,5R)-5-(4-amino-2-chloro-7H-pyrrolo[2,3-d]pyrimidin-7-yl)-2-ethynyl(3- |
| | ²H)-2-(hydroxymethyl)-tetrahydrofuran-3-ol |
| 7 | [(2R,3S,5R)-5-(6-amino-2-fluoro-9H-purin-9-yl)-3-oxhydryl-2-ethynyloxolan-2- |
| | yl](²H)-methanol |
| 8 | [(2R,3S,5R)-5-(6-amino-2-fluoro-9H-purin-9-yl)-3-oxhydryl-2-ethynyloxolan-2- |
| | yl](²H₂)methanol |
| 9 | (2R,3S,5R)-5-(6-amino-2-fluoro-9H-purin-9-yl)-2-(²H)ethynyl-2- |
| | (hydroxymethyl)oxolan-3-ol |
| 10 | (2R,3S,5R)-5-{4-amino-2-chloro-7H-pyrrolo[2,3-d]pyrimidin-7-yl}-2-ethynyl-2- |
| | [hydroxy(²H)methyl]oxolan-3-ol |
| 11 | (2R,3S,5R)-5-{4-amino-2-chloro-7H-pyrrolo[2,3-d]pyrimidin-7-yl}-2-ethynyl-2- |
| | [hydroxy(²H₂)methyl]oxolan-3-ol |
| 12 | (2R,3S,5R)-5-{4-amino-2-chloro-7H-pyrrolo[2,3-d]pyrimidin-7-yl}-2-(²H)ethynyl- |
| | 2-(hydroxymethyl)oxolan-3-ol |
| 13 | (2R,3S,5R)-5-(6-amino-2-fluoro-9H-purin-9-yl)-2-ethynyl-2- |
| | (hydroxymethyl)tetrahydrofuran-5-²H-3-ol |
| 14 | (2R,3S,5R)-5-(4-amino-2-chloro-7H-pyrrolo[2,3-d]pyrimidin-7-yl)-2-ethynyl-2- |
| | (hydroxymethyl)tetrahydrofuran-5-²H-3-ol |

### Viking Assay/CTG

### Viking Assay:

Assessing antiviral potency in a multiple round HIV-1 infection assay. HIV-1 replication was monitored using MT4-gag-GFP clone D3 (hereafter designate MT4-GFP), which are MT-4 cells modified to harbor a GFP reporter gene, the expression of which is dependent on the HIV-1 expressed proteins tat and rev. Productive infection of an MT4-GFP cell with HIV-1 results in GFP expression approximately 24 h post-infection.

MT4-GFP cells were maintained at 37° C./5% CO₂/90% relative humidity in RPMI 1640 supplemented with 10% fetal bovine serum, 100 U/mL penicillin/streptomycin, and 400 μg/mL G418 to maintain the reporter gene. For infections, MT4-GFP cells were placed in the same medium lacking G418 and infected overnight with H9IIIB virus at an approximate multiplicity of infection of 0.01 in the same incubation conditions. Cells were then washed and re-suspended in either RPMI 1640 containing 10% or 50% normal human serum at 1.6×10⁵ cells/mL. Compound plates were prepared by dispensing compounds dissolved in DMSO into wells of 384 well poly D lysine-coated plates (0.2 μl/well) using an ECHO acoustic dispenser. Each compound was tested in a 10 point serial 3-fold dilution (typical final concentrations: 8.4 μM-0.43 nM). Controls included no inhibitor (DMSO only) and a combination of three antiviral agents (efavirenz, indinavir, and an integrase strand transfer inhibitor at final concentrations of 4 μM each). Cells were added (50 μL/well) to compound plates and the infected cells were maintained at 37° C./5% CO₂/90% relative humidity.

Infected cells were quantified at two time points, ˜48 h and ˜72 h post-infection, by counting the number of green cells in each well using an Acumen eX3 scanner. The increase in the number of green cells over ˜24 h period gives the reproductive ratio, R₀, which is typically 5-15 and has been shown experimentally to be in logarithmic phase (data not shown). Inhibition of R₀ is calculated for each well, and IC₅₀s determined by non-linear 4-parameter curve fitting.

### CTG Assay:

### Assessing Cytotoxicity in CellTiter-Glo Luminescent Cell Viability Assay (CTG).

MT4-GFP cells were seeded in RPMI 1640 supplemented with 10% fetal bovine serum, 100 U/mL penicillin/streptomycin overnight at 37° C./5% CO₂/90% relative humidity. Cells were then washed and resuspended in RPMI 1640 containing 10% normal human serum at a density of 0.8×10⁵ cells/mL. Compound plates were prepared by dispensing compounds dissolved in DMSO into wells of 384 well solid black plates (Corning 3571) using an ECHO acoustic dispenser (0.2 μl/well). Each compound was tested in a 10 point serial 3-fold dilution (final concentrations: 8.4 μM-0.43 nM). Controls included DMSO. Cells were added (50 μL/well) to compound plates and were maintained at 37° C./5% CO₂/90% relative humidity. CTG reagent (Promega, G7573) was added to the cell plates after 48 h incubation according to the manufacturer's description. Luminescence signals were recorded on EnVision plate reader (PerkinElmer). CC₅₀s were determined by non-linear 4-parameter curve fitting. The resulting data is shown in Table 3 with the marketed HIV nucleoside reverse transcriptase inhibitor AZT (azidothymidine, zidovudine) included as a control.

TABLE 4

| | | Viking, IC₅₀ | |
|---|---|---|---|
| | Structure | (10%NHS)(nM) | CTG (μM) |
| AZT | 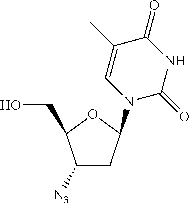 [SMILES: Cc1cn([C@H]2C[C@H](N=[N+]=[N-])[C@@H](CO)O2)c(=O)[nH]c1=O] | 37 | >8.4 |
| Example | | Viking, IC₅₀ | |
| No. | Structure | (10%NHS)(nM) | CTG (μM) |
| 1 | 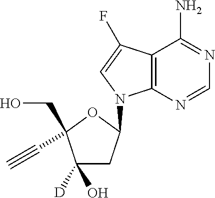 [SMILES: [2H][C@@]1(O)C[C@H](n2cc(F)c3c(N)ncnc32)O[C@]1(C#C)CO] | 2311 | >42.0 |
| 2 | 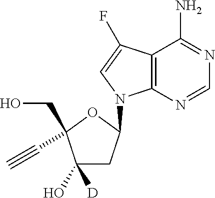 [SMILES: [2H][C@]1(O)C[C@H](n2cc(F)c3c(N)ncnc32)O[C@]1(C#C)CO] | 3.1 | 31.9 |
| 3 | 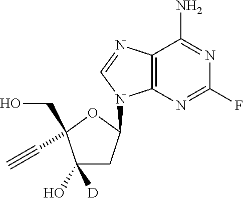 [SMILES: [2H][C@]1(O)C[C@H](n2cnc3c(N)nc(F)nc32)O[C@]1(C#C)CO] | 1.2 | >42.0 |
| 4 | 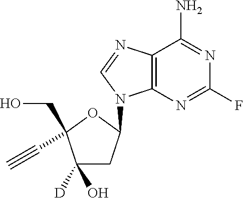 [SMILES: [2H][C@@]1(O)C[C@H](n2cnc3c(N)nc(F)nc32)O[C@]1(C#C)CO] | 1194 | >42.0 |
| 5 | 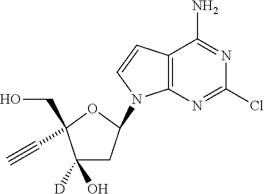 [SMILES: [2H][C@@]1(O)C[C@H](n2ccc3c(N)nc(Cl)nc32)O[C@]1(C#C)CO] | >42000 | >42.0 |
| 6 | 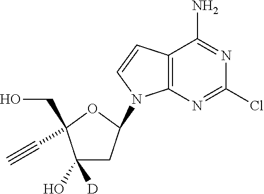 [SMILES: [2H][C@]1(O)C[C@H](n2ccc3c(N)nc(Cl)nc32)O[C@]1(C#C)CO] | 10.6 | >42.0 |
| 7 | 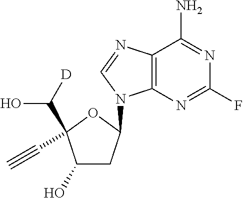 [SMILES: [2H]C(O)[C@@]1(C#C)O[C@@H](n2cnc3c(N)nc(F)nc32)C[C@@H]1O] | 0.7 | >42.0 |
| 8 | 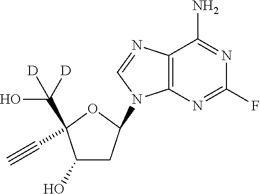 [SMILES: [2H]C([2H])(O)[C@@]1(C#C)O[C@@H](n2cnc3c(N)nc(F)nc32)C[C@@H]1O] | 1.1 | >42.0 |
| 9 | 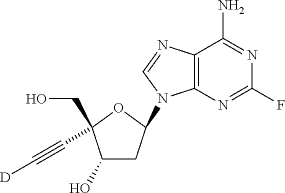 [SMILES: [2H]C#C[C@]1(CO)O[C@@H](n2cnc3c(N)nc(F)nc32)C[C@@H]1O] | 0.6 | >42.0 |
| 10 | 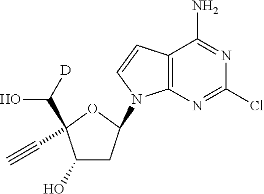 [SMILES: [2H]C(O)[C@@]1(C#C)O[C@@H](n2ccc3c(N)nc(Cl)nc32)C[C@@H]1O] | 5.5 | >42.0 |
| 11 | 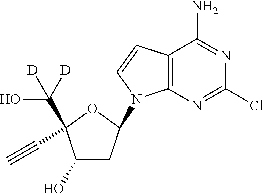 [SMILES: [2H]C([2H])(O)[C@@]1(C#C)O[C@@H](n2ccc3c(N)nc(Cl)nc32)C[C@@H]1O] | 5.6 | >42.0 |
| 12 | 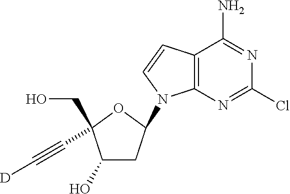 [SMILES: [2H]C#C[C@]1(CO)O[C@@H](n2ccc3c(N)nc(Cl)nc32)C[C@@H]1O] | 4.6 | >42.0 |
| 13 | 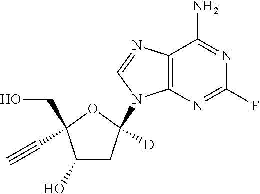 [SMILES: [2H][C@]1(n2cnc3c(N)nc(F)nc32)C[C@H](O)[C@@](C#C)(CO)O1] | 0.2 | >42.0 |
| 14 | 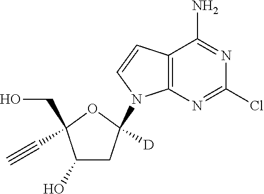 [SMILES: [2H][C@]1(n2ccc3c(N)nc(Cl)nc32)C[C@H](O)[C@@](C#C)(CO)O1] | 0.3 | >42.0 |

While the foregoing specification teaches the principles of the present invention, with examples provided for the purpose of illustration, the practice of the invention encompasses all of the usual variations, adaptations and/or modifications that come within the scope of the following claims. Recitation or depiction of a specific compound in the claims (i.e., a species) without a specific stereoconfiguration designation, or with such a designation for less than all chiral centers, is intended to encompass the racemate, racemic mixtures, each individual enantiomer, a diastereoisomeric mixture and each individual diastereomer of the compound where such forms are possible due to the presence of one or more asymmetric centers. All publications, patents and patent applications cited herein are incorporated by reference in their entirety into the disclosure.

1. A compound of structural Formula I 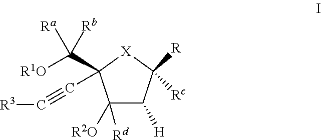
or a pharmaceutically acceptable salt thereof, wherein:
R is 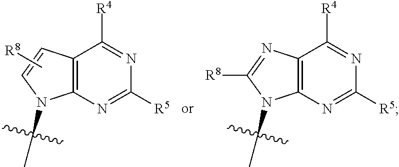
X is O, S, CH₂ or CF₂;
R¹ is —H, —C(O)R⁶, —C(O)OR⁶, —C(O)N(R⁶)₂, 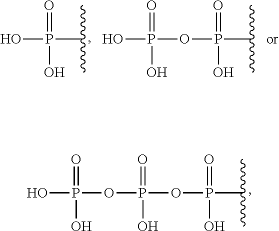
or a pro-drug modification of the mono-, di- or triphosphate;
R² is —H, —C(O)R^{6a}, —C(O)OR⁶a or —C(O)N(R^{6a})₂;
R³, R^{a}, R^{b}, R^{c} and R^{d} are each independently —H or -D, provided that at least one of R³, R^{a}, R^{b}, R^{c} and R^{d} is -D;
D is deuterium;
R⁴ is —H, —C₁-C₆ alkyl, —C₂-C₆ alkenyl, —C₂-C₆ alkynyl, —C₁-C₆ haloalkyl, —C₃-C₇ cycloalkyl, 5- or 6-membered monocyclic heteroaryl, a 9- or 10-membered bicyclic heteroaryl, halo, —CN, —NO₂, —N(R^{X})₂, —NH(C₁-C₆alkylene)-(5- or 6-membered monocyclic heteroaryl), —NH(C₁-C₆ alkylene)-(9- or 10-membered bicyclic heteroaryl), aryl, —NHC(O)OR^{6b}, —N(C(O)OR^{6b})₂, —NHC(O)N(R^{6b})₂, or —NHC(O)R^{6b}, wherein each of said —C₁-C₆ alkyl group, said —C₂-C₆ alkenyl group or said —C₂-C₆ alkynyl group can be optionally substituted with halo;
R⁵ is —H, —C₁-C₆ alkyl, —C₂-C₆ alkenyl, —C₂-C₆ alkynyl, —C₁-C₆ haloalkyl, —C₃-C₇ cycloalkyl, 5- or 6-membered monocyclic heteroaryl, a 9- or 10-membered bicyclic heteroaryl, halo, —OR^{X}, —CN, —NO₂, —N(R^{X})₂, —NH(C₁-C₆alkylene)-(5- or 6-membered monocyclic heteroaryl), —NH(C₁-C₆ alkylene)-(9- or 10-membered bicyclic heteroaryl), aryl, —NHC(O)OR^{6b}, —N(C(O)OR^{6b})₂, —NHC(O)N(R^{6b})₂, or —NHC(O)R^{6b}, wherein each of said —C₁-C₆ alkyl group, said —C₂-C₆ alkenyl group or said —C₂-C₆ alkynyl group can be optionally substituted with halo;
R⁶, R^{6a} and R^{6b} are each independently selected at each occurrence from —H, —C₁-C₆ alkyl, —C₁-C₆ haloalkyl, —(C₁-C₃ alkylene)ₘ-(C₃-C₇ cycloalkyl), —(C₁-C₃ alkylene)ₘ-(aryl), —(C₁-C₃ alkylene)ₘ-(4 to 7-membered heterocycloalkyl), —(C₁-C₃ alkylene)ₘ-(5- or 6-membered monocyclic heteroaryl) or —(C₁-C₃ alkylene)ₘ-(9- or 10-membered bicyclic heteroaryl), wherein each of said —C₁-C₆ alkyl, said C₃-C₇ cycloalkyl group, said aryl group, said 4 to 7-membered heterocycloalkyl group, said -(5- or 6-membered monocyclic heteroaryl group or said 9- or 10-membered bicyclic heteroaryl group can be optionally substituted with R⁷;
m is an integer selected from 0 (zero) or 1;
R⁷ represents from one to five substituent groups, each independently selected from —C₁-C₆alkyl, C₂-C₆ alkenyl, C₂-C₆ alkynyl, C₁-C₆ haloalkyl, aryl, or a 5-6-member heteroaryl;
R⁸ is —H, halo, —C₁-C₆ alkyl, —C₁-C₆ haloalkyl, —CN, —ORS' or —N(R^{Y})₂;
R^{X} is independently selected at each occurrence from —H, —C₁-C₆ alkyl, —C₁-C₆ haloalkyl, aryl, or 5- or 6-membered monocyclic heteroaryl;
or when either or both of R⁴ or R⁵ is —N(R^{X})₂, each R^{X} may optionally be joined together with the nitrogen to which they are both attached to form a 5- or 6-membered monocyclic heteroaryl or 9- or 10-membered bicyclic heteroaryl; and
R^{Y} is —H, —C₁-C₆ alkyl or —C₁-C₆ haloalkyl.

2. The compound of claim 1 or a pharmaceutically acceptable salt thereof wherein X is O, S or CH₂.

3. The compound of claim 2 or a pharmaceutically acceptable salt thereof wherein R¹ is —H or

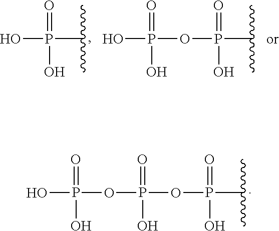

4. The compound of claim 3 or a pharmaceutically acceptable salt thereof wherein one of R³, R^{a}, R^{b}, R^{c} and R^{d} is -D.

5. The compound of claim 3 or a pharmaceutically acceptable salt thereof wherein R^{a} and R^{b} are both -D.

6. The compound of claim 3 or a pharmaceutically acceptable salt thereof wherein R² is —H.

7. The compound of claim 6 or a pharmaceutically acceptable salt thereof wherein R³ is —H.

8. The compound of claim 3 or a pharmaceutically acceptable salt thereof wherein R⁴ is —N(R^{X})₂.

9. The compound of claim 8 or a pharmaceutically acceptable salt thereof wherein R⁵ is —H, halo, —C₁-C₆ alkyl, —OR^{X}, —N(R^{X})₂.

10. The compound of claim 9 or a pharmaceutically acceptable salt thereof wherein R⁶, R^{6a} and R^{6b} are each independently selected at each occurrence from —H, —C₁-C₆ alkyl, —C₁-C₆ haloalkyl, phenyl, or a 5- or 6-membered monocyclic heteroaryl.

11. The compound of claim 10 or a pharmaceutically acceptable salt thereof wherein R⁷ is —C₁-C₆ alkyl, aryl phenyl or a 5-6 member monocyclic heteroaryl.

12. The compound of claim 11 or a pharmaceutically acceptable salt thereof wherein R⁸ is —H, halo, —C₁-C₃ alkyl, —C₁-C₃ haloalkyl, —CN, —OR^{Y}, or —N(R^{Y})₂.

13. The compound of claim 1 or a pharmaceutically acceptable salt thereof wherein:
X is O;
R¹ is —H;
R² is —H;
R³, R^{a}, R^{b}, R^{c} and R^{d} are each independently —H or -D, provided that at least one of R³, R^{a}, R^{b}, R^{c} and R^{d} is -D;
D is deuterium;
R⁴ is —NH₂;
R⁵ is —H or halo; and
R⁸ is —H or halo.

14. The compound of claim 1 that is:

| | |
|---|---|
| 1 | (2R,3R,5R)-5-{4-amino-5-fluoro-7H-pyrrolo[2,3-d]pyrimidin-7-yl}-2-{[(tert- |
| | butyldiphenylsilyl)oxy]methyl}-2-ethynyl(3-²H)oxolan-3-ol; |
| 2 | (2R,3S,5R)-5-{4-amino-5-fluoro-7H-pyrrolo[2,3-d]pyrimidin-7-yl}-2-ethynyl-2- |
| | (hydroxymethyl)(3-²H)oxolan-3-ol; |
| 3 | (2R,3S,5R)-5-(6-amino-2-fluoro-9H-purin-9-yl)-2-ethynyl-2-(hydroxymethyl)(3- |
| | ²H)oxolan-3-ol; |
| 4 | (2R,3R,5R)-5-(6-amino-2-fluoro-9H-purin-9-yl)-2-ethynyl-2-(hydroxymethyl)(3- |
| | ²H)oxolan-3-ol; |
| 5 | (2R,3R,5R)-5-(4-amino-2-chloro-7H-pyrrolo[2,3-d]pyrimidin-7-yl)-2-ethynyl(3-²H)-2- |
| | (hydroxymethyl)-tetrahydrofuran-3-ol; |
| 6 | (2R,3S,5R)-5-(4-amino-2-chloro-7H-pyrrolo[2,3-d]pyrimidin-7-yl)-2-ethynyl(3-²H)-2- |
| | (hydroxymethyl)-tetrahydrofuran-3-ol; |
| 7 | [(2R,3S,5R)-5-(6-amino-2-fluoro-9H-purin-9-yl)-3-oxhydryl-2-ethynyloxolan-2-yl](²H)- |
| | methanol; |
| 8 | [(2R,3S,5R)-5-(6-amino-2-fluoro-9H-purin-9-yl)-3-oxhydryl-2-ethynyloxolan-2- |
| | yl](²H₂)methanol; |
| 9 | (2R,3S,5R)-5-(6-amino-2-fluoro-9H-purin-9-yl)-2-(²H)ethynyl-2-(hydroxymethyl)oxolan- |
| | 3-ol; |
| 10 | (2R,3S,5R)-5-{4-amino-2-chloro-7H-pyrrolo[2,3-d]pyrimidin-7-yl}-2-ethynyl-2- |
| | [hydroxy(²H)methyl]oxolan-3-ol; |
| 11 | (2R,3S,5R)-5-{4-amino-2-chloro-7H-pyrrolo[2,3-d]pyrimidin-7-yl}-2-ethynyl-2- |
| | [hydroxy(²H₂)methyl]oxolan-3-ol; |
| 12 | (2R,3S,5R)-5-{4-amino-2-chloro-7H-pyrrolo[2,3-d]pyrimidin-7-yl}-2-(²H)ethynyl-2- |
| | (hydroxymethyl)oxolan-3-ol; |
| 13 | (2R,3S,5R)-5-(6-amino-2-fluoro-9H-purin-9-yl)-2-ethynyl-2- |
| | (hydroxymethyl)tetrahydrofuran-5-²H-3-ol; or |
| 14 | (2R,3S,5R)-5-(4-amino-2-chloro-7H-pyrrolo[2,3-d]pyrimidin-7-yl)-2-ethynyl-2- |
| | (hydroxymethyl)tetrahydrofuran-5-²H-3-ol; |

or a pharmaceutically acceptable salt thereof.

15. A pharmaceutical composition comprising an effective amount of a compound of claim 1, or a pharmaceutically acceptable salt thereof, and a pharmaceutically acceptable carrier.

16. A pharmaceutical composition comprising an effective amount of a compound of claim 1, or a pharmaceutically acceptable salt thereof, and a pharmaceutically acceptable carrier and further comprising an effective amount of an anti-HIV agent selected from an HIV antiviral agent, an immunomodulator, or anti-infective agent.

17. The pharmaceutical composition of claim 16, wherein the HIV antiviral agent is an HIV protease inhibitor, HIV reverse transcriptase inhibitor, HIV integrase inhibitor, HIV fusion inhibitor, HIV entry inhibitor, or HIV maturation inhibitor.

18. A method for the treatment or prophylaxis of infection by HIV or for the treatment, prophylaxis, or delay in the onset of AIDS in a subject in need thereof, which comprises administering to the subject an effective amount of the compound of claim 1, or a pharmaceutically acceptable salt thereof.

19. The method of claim 18 further comprising administering to the subject an effective amount of one or more anti-HIV agent selected from: abacavir, abacavir sulfate, abacavir+lamivudine, abacavir+lamivudine+zidovudine, amprenavir, atazanavir, atazanavir sulfate, AZT, capravirine, darunavir, dideoxycytidine, dideoxyinosine, delavirdine, delavirdine mesylate, dolutegravir, doravirine, efavirenz, efavirenz+emtricitabine+tenofovir disoproxil fumarate, 4′-ethynyl-2-fluoro-2′-deoxyadenosine, elvitegravir emtricitabine, emtricitabine+tenofovir disoproxil fumarate, emivirine, enfuvirtide, etravirine, fosamprenavir calcium, indinavir, indinavir sulfate, lamivudine, lamivudine+zidovudine, lopinavir, lopinavir+ritonavir, maraviroc, nelfinavir, nelfinavir mesylate, nevirapine, PPL-100, raltegravir, rilpivirine ritonavir, saquinavir, saquinavir mesylate, stavudine, tenofovir disoproxil fumarate, tenofovir alafenamide fumarate, tipranavir, vicriviroc or vicriviroc mealeat.

20. (canceled)
